(19) United States
(12) Patent Application Publication
(10) Pub. No.: US 2012/0053168 A1
(43) Pub. Date: Mar. 1, 2012

(54) FUSED BENZOAZEPINES AS NEURONAL NICOTINIC ACETYLCHOLINE RECEPTOR LIGANDS

(75) Inventors: Balwinder Singh Bhatti, Winston-Salem, NC (US); Timothy J. Cuthbertson, Winston-Salem, NC (US); Anatoly Mazurov, Greensboro, NC (US); Joseph Pike Mitchener, Winston-Salem, NC (US); Julio A. Munoz, Walnut Cove, NC (US); Srinivasa V. Murthy, Winston-Salem, NC (US); Yunde Xiao, Clemmons, NC (US); Daniel Yohannes, Winston-Salem, NC (US)

(73) Assignee: Targacept, Inc., Winson-Salem, NC (US)

(21) Appl. No.: 13/148,385

(22) PCT Filed: Feb. 16, 2010
(86) PCT No.: PCT/US10/24294

§ 371 Date: Nov. 14, 2011

Related U.S. Application Data
(60) Provisional application No. 61/153,138, filed on Feb. 17, 2009.

(51) Int. Cl. A61K 31/55; C07D 471/04; C07D 223/16; A61P 25/28; A61P 25/00; A61P 9/10; A61P 25/18; A61P 25/16; A61P 25/14; A61P 29/00; A61P 25/08; A61P 25/22; A61P 25/24; A61P 3/04; A61P 25/30; A61P 25/32; A61P 25/34; A61P 25/04; C07D 487/04

(52) U.S. Cl. 514/215; 540/578; 540/580; 540/594

(57) ABSTRACT

The present invention relates to compounds that bind to and modulate the activity of neuronal nicotinic acetylcholine receptors, to processes for preparing these compounds, to pharmaceutical compositions containing these compounds, and to methods of using these compounds for treating a wide variety of conditions and disorders, including those associated with dysfunction of the central nervous system (CNS).

### FIELD OF THE INVENTION

The present invention relates to compounds that bind to and modulate the activity of neuronal nicotinic acetylcholine receptors, to processes for preparing these compounds, to pharmaceutical compositions containing these compounds, and to methods of using these compounds for treating a wide variety of conditions and disorders, including those associated with dysfunction of the central nervous system (CNS).

### BACKGROUND OF THE INVENTION

The therapeutic potential of compounds that target neuronal nicotinic receptors (NNRs), also known as nicotinic acetylcholine receptors (nAChRs), has been the subject of several reviews. See, for example, Breining et al., Ann. Rep. Med. Chem. 40: 3 (2005), Hogg and Bertrand, Curr. Drug Targets: CNS Neurol. Disord. 3: 123 (2004), Suto and Zacharias, Expert Opin. Ther. Targets 8: 61 (2004), Dani et al., Bioorg. Med. Chem. Lett. 14: 1837 (2004), Bencherif and Schmitt, Curr. Drug Targets: CNS Neurol. Disord. 1: 349 (2002). Among the kinds of indications for which NNR ligands have been proposed as therapies are cognitive disorders, including Alzheimer's disease, attention deficit disorder, and schizophrenia (Newhouse et al., Curr. Opin. Pharmacol. 4: 36 (2004), Levin and Rezvani, Curr. Drug Targets: CNS Neurol. Disord. 1: 423 (2002), Graham et al., Curr. Drug Targets: CNS Neurol. Disord. 1: 387 (2002), Ripoll et al., Curr. Med. Res. Opin. 20(7): 1057 (2004), and McEvoy and Allen, Curr. Drug Targets: CNS Neurol. Disord. 1:

433 (2002)); pain and inflammation (Decker et al., Curr. Top. Med. Chem. 4(3): 369 (2004), Vincler, Expert Opin. Invest. Drugs 14(10): 1191 (2005), Jain, Curr. Opin. Inv. Drugs 5: 76 (2004), Miao et al., Neuroscience 123: 777 (2004)); depression and anxiety (Shytle et al., Mol. Psychiatry 7: 525 (2002), Damaj et al., Mol. Pharmacol. 66: 675 (2004), Shytle et al., Depress. Anxiety 16: 89 (2002)); neurodegeneration (O'Neill et al., Curr. Drug Targets: CNS Neurol. Disord. 1: 399 (2002), Takata et al., J. Pharmacol. Exp. Ther. 306: 772 (2003), Marrero et al., J. Pharmacoi. Exp. Ther. 309: 16 (2004)); Parkinson's disease (Jonnala and Buccafusco, J. Neurosci. Res. 66: 565 (2001)); addiction (Hansen and Mark, Psychopharmacol. 194(1): 53-61 (2007), Steensland et al., PNAS 104(30): 12518-12523 (2007), Coe et al., Bioorg. Med. Chem. Lett. 15(22): 4889 (2005)); obesity (Li et al., Curr. Top. Med. Chem. 3: 899 (2003)); and Tourette's syndrome (Sacco et al., J. Psychopharmacol. 18(4): 457 (2004), Young et al., Clin. Ther. 23(4): 532 (2001))

There exists a heterogeneous distribution of nAChR subtypes in both the central and peripheral nervous systems. For instance, the nAChR subtypes which are predominant in vertebrate brain are α4β2, α7, and α3β2, whereas those which predominate at the autonomic ganglia are α3β4 and those of neuromuscular junction are α1β1δγ and α1β1δε (see Dwoskin et al., Exp. Opin. Ther. Patents 10: 1561 (2000) and Holliday et al. J. Med. Chem. 40(26), 4169 (1997)).

A limitation of some nicotinic compounds is that they are associated with various undesirable side effects which can occur, for example, by stimulating muscle and ganglionic receptors. Therefore, there is a need to have compounds, compositions, and methods for preventing or treating various conditions or disorders where the compounds exhibit a high enough degree of nAChR subtype specificity to elicit a beneficial effect, without significantly affecting those receptor subtypes which have the potential to induce undesirable side effects, including, for example, appreciable activity at cardiovascular and skeletal muscle sites.

### SUMMARY OF THE INVENTION

One aspect of the present invention includes a compound of Formula 1 or Formula 2:

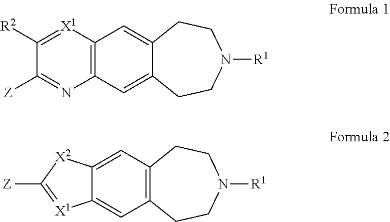

wherein, each X¹ independently is N or CR¹⁰; X² is NR¹⁰ or O; each Z independently is H, R¹⁰, OR¹⁰, NHR¹⁰, NR¹⁰R¹¹, or halogen; each R¹ independently is H or C₁₋₆ alkyl; each R² independently is H, C₁₋₆ alkyl, aryl, or heteroaryl; which aryl and heteroaryl groups can optionally be substituted with one or more of C₁₋₆ alkyl, halogen, hydroxyl, C₁₋₆ alkoxy, amino, or C₁₋₆ haloalkyl; and each of R¹⁰ or R¹¹ independently is H, C₁₋₆ alkyl, aryl, or heteroaryl; which aryl and heteroaryl groups can optionally be substituted with one or more of C₁₋₆ alkyl, halogen, hydroxyl, C₁₋₆ alkoxy, amino, or C₁₋₆ haloalkyl; or a pharmaceutically acceptable salt thereof.

In certain embodiments, the invention includes compounds that are tautomeric to those of Formulae 1 or 2, such as those of Formula 3, when Z is OH or Formula 4, when X¹ is N and Z is OH, respectively:

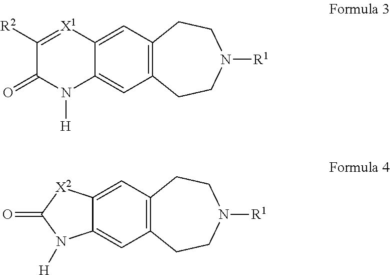

Similarly, although not referred to as tautomeric due to the variability in R¹⁰, the present invention includes alternative structural isomers such as those of Formula 5, when Z is OR¹⁰ or Formula 6, when X¹ is N and Z is OR¹⁰, respectively:

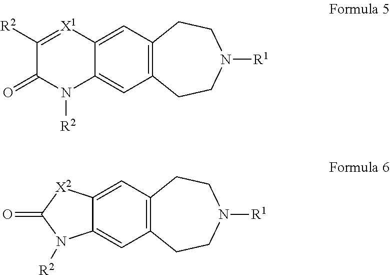

Another aspect of the present invention includes novel intermediates and synthetic processes. The present invention includes a compound 7,8-diamino-2,3,4,5-tetrahydro-1H-benzo[d]azepine, also known as 7,8-diamino-1,2,4,5-tetrahydro-3H-3-benzazepine, or a 3-N-protected derivative thereof, such as 7,8-diamino-3-trifluoroacetyl-2,3,4,5-tetrahydro-1H-benzo[d]azepine or tert-butyl 7,8-diamino-1,2,4,5-tetrahydro-3H-3-benzazepine-3-carboxylate.

Another aspect of the present invention includes a method of making a 3-N-protected-7,8-diamino-2,3,4,5-tetrahydro-1H-benzo[d]azepine, comprising the steps of:
i) nitrating 2,3,4,5-tetrahydro-1H-3-benzazepine to form 7-nitro-2,3,4,5-tetrahydro-1H-3-benzazepine;
ii) converting the 7-nitro-2,3,4,5-tetrahydro-1H-3-benzazepine into a suitable 3-N-protected derivative;
iii) reducing the 7-nitro group to form a 7-amino group;
iv) converting 7-amino group to an amide derivative (i.e., acylamino group);
v) nitrating the 7-acylamino compound to form the 7-acylamino-8-nitro compound;
vi) reducing 8-nitro group to form an 8-amino group; and
vii) hydrolyzing the acyl group from the 7-position amine.
   In one embodiment, the synthetic method includes condensation of a 3-N-protected-7,8-diamino-2,3,4,5-tetrahydro-1H-benzo[d]azepine with another reagent or reagents. In a further embodiment, the method further includes the step of removing the protecting group from the 3-position amino group.

The compounds of the present invention bind with high affinity to NNRs of the α4β2 subtype found in the CNS, yet exhibit low affinity for the α7 subtype of the CNS and the peripheral muscle and ganglionic receptor subtypes. The present invention also relates to pharmaceutically acceptable salts prepared from these compounds.

The present invention includes pharmaceutical compositions comprising a compound of the present invention or a pharmaceutically acceptable salt thereof. The pharmaceutical compositions of the present invention can be used for treating or preventing a wide variety of conditions or disorders, and particularly those disorders characterized by dysfunction of nicotinic cholinergic neurotransmission or the degeneration of the nicotinic cholinergic neurons.

The present invention includes a method for treating or preventing disorders and dysfunctions, such as CNS disorders and dysfunctions, inflammation, inflammatory response associated with bacterial and/or viral infection, pain, neovascularization, or other disorders described in further detail herein. Additionally, these compounds may also have utility as diagnostic agents and in receptor binding studies as described herein. The methods involve administering to a subject a therapeutically effective amount of a compound of the present invention, including a salt thereof, or a pharmaceutical composition that includes such compounds.

The present invention also includes combinations of aspects, embodiments, and preferences as herein described.

The foregoing and other aspects of the present invention are explained in further detail in the detailed description and examples set forth below.

### BRIEF DESCRIPTION OF THE FIGURES

FIG. 1 graphically depicts the ability of 7,8,9,10-tetrahydro-6H-azepino[4,5-g]quinoxaline, herein also referred to as Compound A, to improve cognitive performances of normal rats was evaluated using a Novel Object Recognition (NOR) model.

FIG. 2 graphically depicts the ability of Compound A to improve cognitive performances of animals pharmacologically impaired with scopolamine using the radial arm maze (RAM) task (left panel) and the Morris water maze (MWM) task (right panel).

### DETAILED DESCRIPTION OF THE INVENTION

### I. Compounds

One aspect of the present invention includes a compound of Formula 1 or Formula 2:

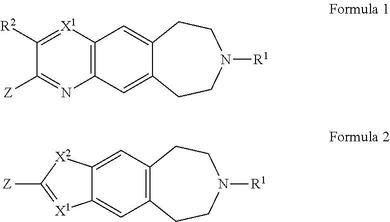

wherein, each X¹ independently is N or CR¹⁰; X² is NR¹⁰ or O; each Z independently is H, R¹⁰, OR¹⁰, NHR¹⁰, NR¹⁰R¹¹, or halogen; each R¹ independently is H or C₁₋₆ alkyl; each R² independently is H, C₁₋₆ alkyl, aryl, or heteroaryl; which aryl and heteroaryl groups can optionally be substituted with one or more of C₁₋₆ alkyl, halogen, hydroxyl, C₁₋₆ alkoxy, amino, or C₁₋₆ haloalkyl; and each of R¹⁰ or R¹¹ independently is H, C₁₋₆ alkyl, aryl, or heteroaryl; which aryl and heteroaryl groups can optionally be substituted with one or more of C₁₋₆ alkyl, halogen, hydroxyl, C₁₋₆ alkoxy, amino, or C₁₋₆ haloalkyl; or a pharmaceutically acceptable salt thereof.

In one embodiment, the compound is of Formula 1 and, in a further embodiment, the compound is a tautomer or other structural isomer. In another embodiment, the compound of Formula 2 and, in a further embodiment, the compound is a tautomer or other structural isomer.

In one embodiment, X¹ is N.

In one embodiment, X¹ is CR¹⁰.

In one embodiment, R¹ is H.

In one embodiment, R¹ is C₁₋₆ alkyl.

In one embodiment, the present invention includes a compound selected from:

7,8,9,10-tetrahydro-6H-azepino[4,5-g]quinoxaline; 8-methyl-7,8,9,10-tetrahydro-6H-azepino[4,5-g]quinoxaline; 2-methyl-7,8,9,10-tetrahydro-6H-azepino[4,5-g]quinoxaline; 2,3-dimethyl-7,8,9,10-tetrahydro-6H-azepino[4,5-g]quinoxaline; 2-methyl-3-ethyl-7,8,9,10-tetrahydro-6H-azepino[4,5-g]quinoxaline; 2,3-diethyl-7,8,9,10-tetrahydro-6H-azepino[4,5-g]quinoxaline; 2,8-dimethyl-7,8,9,10-tetrahydro-6H-azepino[4,5-g]quinoxaline; 7,8,9,10-tetrahydro-6H-azepino[4,5-g]quinoxalin-2(1H)-one; 2-chloro-7,8,9,10-tetrahydro-6H-azepino[4,5-g]quinoxaline; 2-chloro-8-methyl-7,8,9,10-tetrahydro-6H-azepino[4,5-g]quinoxaline; 2-methoxy-7,8,9,10-tetrahydro-6H-azepino[4,5-g]quinoxaline; 2-methoxy-8-methyl-7,8,9,10-tetrahydro-6H-azepino[4,5-g]quinoxaline; 2-(N-methylamino)-7,8,9,10-tetrahydro-6H-azepino[4,5-g]quinoxaline; 2-(N,N-dimethylamino)-7,8,9,10-tetrahydro-6H-azepino[4,5-g]quinoxaline; 2-(N-benzylamino)-7,8,9,10-tetrahydro-6H-azepino[4,5-g]quinoxaline; 2-(3-pyridinyl)-7,8,9,10-tetrahydro-6H-azepino[4,5-g]quinoxaline; 7,8,9,10-tetrahydro-6H-azepino[4,5-g]quinoline; 8-methyl-7,8,9,10-tetrahydro-6H-azepino[4,5-g]quinoline; 2-methyl-7,8,9,10-tetrahydro-6H-azepino[4,5-g]quinoline; 2,3-dimethyl-7,8,9,10-tetrahydro-6H-azepino[4,5-g]quinoline; 2,8-dimethyl-7,8,9,10-tetrahydro-6H-azepino[4,5-g]quinoline; 7,8,9,10-tetrahydro-6H-azepino[4,5-g]quinolin-2(1H)-one; 2-chloro-7,8,9,10-tetrahydro-6H-azepino[4,5-g]quinoline; 2-chloro-8-methyl-7,8,9,10-tetrahydro-6H-azepino[4,5-g]quinoline; 2-methoxy-7,8,9,10-tetrahydro-6H-azepino[4,5-g]quinoline; 2-methoxy-8-methyl-7,8,9,10-tetrahydro-6H-azepino[4,5-g]quinoline; 2-(N-methylamino)-7,8,9,10-tetrahydro-6H-azepino[4,5-g]quinoline; 2-(N,N-dimethylamino)-7,8,9,10-tetrahydro-6H-azepino[4,5-g]quinoline; 2-phenyl-7,8,9,10-tetrahydro-6H-azepino[4,5-g]quinoline; 2-(3-pyridinyl)-7,8,9,10-tetrahydro-6H-azepino[4,5-g]quinoline; 1,5,6,7,8,9-hexahydroimidazo[4,5-h][3]benzazepine; 1-methyl-1,5,6,7,8,9-hexahydroimidazo[4,5-h][3]benzazepine; 2-methyl-1,5,6,7,8,9-hexahydroimidazo[4,5-h][3]benzazepine; 1,7-dimethyl-1,5,6,7,8,9-hexahydroimidazo[4,5-h][3]benzazepine; 2,7-dimethyl-1,5,6,7,8,9-hexahydroimidazo[4,5-h][3]benzazepine; 1,2-dimethyl-1,5,6,7,8,9-hexahydroimidazo[4,5-h][3]benzazepine; 2-methyl-1-phenyl-1,5,6,7,8,9-hexahydroimidazo[4,5-h][3]benzazepine; 3,5,6,7,8,9-hexahydroimidazo[4,5-h][3]benzazepin-2(1H)-one; 1-methyl-3,5,6,7,8,9-hexahydroimidazo[4,5-h][3]benzazepin-2(1H)-one; 1-phenyl-3,5,6,7,8,9-hexahydroimidazo[4,5-h][3]benzazepin-2(1H)-one; 1,7-dimethyl-3,5,6,7,8,9-hexahydroimidazo[4,5-h][3]benzazepin-2(1H)-one; and 7-methyl-1-phenyl-3,5,6,7,8,9-hexahydroimidazo[4,5-h][3]benzazepin-2(1H)-one,;
or a pharmaceutically acceptable salt thereof.

One aspect of the present invention includes a compound of the present invention or a pharmaceutically acceptable salt thereof for use as an active therapeutic substance. Another aspect of the present invention includes a pharmaceutical composition comprising a compound of the present invention or a pharmaceutically acceptable salt thereof and a pharmaceutically acceptable carrier. Another aspect of the present invention includes a method for the treatment or prevention of a disease or condition mediated by a neuronal nicotinic receptor comprising the administration of a compound of the present invention or a pharmaceutically acceptable salt thereof, or a pharmaceutical composition of the present invention. In one embodiment, the neuronal nicotinic receptor is of the α4β2 subtype. In one embodiment, the disease or condition is a CNS disorder. In another embodiment, the disease or condition is inflammation or an inflammatory response associated with one or more of a bacterial or viral infection. In another embodiment, the disease or condition is pain. In another embodiment, the disease or condition is neovascularization. In another embodiment, the disease or condition is another disorder described herein.

In another aspect, the compounds of the present invention are administered to a mammal to serve as diagnostic agents. In another embodiment, the compounds are used in receptor binding studies.

The scope of the present invention is described in further detail herein and includes all combinations of aspects and embodiments.

The following definitions are meant to clarify, but not limit, the terms defined. If a particular term used herein is not specifically defined, such term should not be considered indefinite. Rather, terms are used within their accepted meanings.

As used herein, the term “pharmaceutically acceptable” refers to carrier(s), diluent(s), excipient(s) or salt forms of the compounds of the present invention that are compatible with the other ingredients of the formulation and not deleterious to the recipient of the pharmaceutical composition.

As used herein, the term “pharmaceutical composition” refers to a compound of the present invention optionally admixed with one or more pharmaceutically acceptable carriers, diluents, or exipients. Pharmaceutical compositions preferably exhibit a degree of stability to environmental conditions so as to make them suitable for manufacturing and commercialization purposes.

As used herein, the terms “effective amount”, “therapeutic amount”, or “effective dose” refer to an amount of the compound of the present invention sufficient to elicit the desired pharmacological or therapeutic effects, thus resulting in effective prevention or treatment of a disorder. Prevention of the disorder may be manifested by delaying or preventing the progression of the disorder, as well as the onset of the symptoms associated with the disorder. Treatment of the disorder may be manifested by a decrease or elimination of symptoms, inhibition or reversal of the progression of the disorder, as well as any other contribution to the well being of the patient.

The effective dose can vary, depending upon factors such as the condition of the patient, the severity of the symptoms of the disorder, and the manner in which the pharmaceutical composition is administered. Typically, to be administered in an effective dose, compounds are required to be administered in an amount of less than 5 mg/kg of patient weight. Often, the compounds may be administered in an amount from less than about 1 mg/kg patient weight to less than about 100 μg/kg of patient weight, and occasionally between about 10 μg/kg to less than 100 μg/kg of patient weight. The foregoing effective doses typically represent that amount administered as a single dose, or as one or more doses administered over a 24 hours period. For human patients, the effective dose of the compounds may require administering the compound in an amount of at least about 1 mg/24 hr/patient, but not more than about 1000 mg/24 hr/patient, and often not more than about 500 mg/ 24 hr/ patient.

As used throughout this specification, the preferred number of atoms, such as carbon atoms, will be represented by, for example, the phrase “C_{x-y} alkyl,” which refers to an alkyl group, as herein defined, containing the specified number of carbon atoms. Similar terminology will apply for other preferred terms and ranges as well. Thus, for example, C₁₋₆ alkyl represents a straight or branched chain hydrocarbon containing one to six carbon atoms.

As used herein the term “alkyl” refers to a straight or branched chain hydrocarbon, which may be optionally substituted, with multiple degrees of substitution being allowed. Examples of “alkyl” as used herein include, but are not limited to, methyl, ethyl, propyl, isopropyl, isobutyl, n-butyl, tert-butyl, isopentyl, and n-pentyl.

As used herein, the term “aryl” refers to a single benzene ring or fused benzene ring system which may be optionally substituted, with multiple degrees of substitution being allowed. Examples of “aryl” groups as used include, but are not limited to, phenyl, 2-naphthyl, 1-naphthyl, anthracene, and phenanthrene. Preferable aryl rings have five- to ten-members.

As used herein, a fused benzene ring system encompassed within the term “aryl” includes fused polycyclic hydrocarbons, namely where a cyclic hydrocarbon with less than maximum number of noncumulative double bonds, for example where a saturated hydrocarbon ring (cycloalkyl, such as a cyclopentyl ring) is fused with an aromatic ring (aryl, such as a benzene ring) to form, for example, groups such as indanyl and acenaphthalenyl, and also includes such groups as, for non-limiting examples, dihydronaphthalene and tetrahydronaphthalene.

As used herein, the term “heteroaryl” refers to a monocyclic five to seven membered aromatic ring, or to a fused bicyclic aromatic ring system comprising two of such aromatic rings, which may be optionally substituted, with multiple degrees of substitution being allowed. Preferably, such rings contain five- to ten-members. These heteroaryl rings contain one or more nitrogen, sulfur, and/or oxygen atoms, where N-oxides, sulfur oxides, and dioxides are permissible heteroatom substitutions. Examples of “heteroaryl” groups as used herein include, but are not limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzoxazole, benzothiophene, indole, indazole, benzimidazole, imidazopyridine, pyrazolopyridine, and pyrazolopyrimidine.

As used herein the term “halogen” refers to fluorine, chlorine, bromine, or iodine.

As used herein the term “haloalkyl” refers to an alkyl group, as defined herein, that is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as —CF₃.

As used herein the term “alkoxy” refers to a group —OR^{a}, where R^{a} is alkyl as defined above.

As used herein “amino” refers to a group —NR^{a}R^{b}, where each of R^{a} and R^{b} individually is hydrogen, alkyl, alkenyl, alkynyl, cycloalkyl, aryl, heterocylcyl, or heteroaryl. As used herein, when either R^{a} or R^{b} is other than hydrogen, such a group may also be referred to as a “substituted amino” or, for example if R^{a} is H and R^{b} is alkyl, as an “alkylamino,” or is R^{a} is alkyl and R^{b} is alkyl as a “dialkylamino.”

As used herein, the terms “hydroxy” and “hydroxyl” refers to a group —OH.

The compounds of this invention may be made by a variety of methods, including well-known standard synthetic methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1999) Protecting Groups in Organic Synthesis, 3^{rd} Edition, John Wiley & Sons,). These groups are removed at a convenient stage of the compound synthesis using methods that are readily apparent to those skilled in the art. The selection of processes as well as the reaction conditions and order of their execution shall be consistent with the preparation of compounds of the present invention.

The present invention also provides a method for the synthesis of compounds useful as intermediates in the preparation of compounds of the present invention along with methods for their preparation.

The compounds can be prepared according to the methods described below using readily available starting materials and reagents. In these reactions, variants may be employed which are themselves known to those of ordinary skill in this art, but are not mentioned in greater detail.

Unless otherwise stated, structures depicted herein are also meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by the formulae of the present invention, as well as any wholly or partially equilibrated mixtures thereof. The present invention also includes the individual isomers of the compounds represented by the formulas above as mixtures with isomers thereof in which one or more chiral centers are inverted.

When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be effected by any suitable method known in the art. See, for example, Stereochemistry of Organic Compounds (Wiley-Interscience, 1994).

The present invention includes a salt or solvate of the compounds herein described, including combinations thereof such as a solvate of a salt. The compounds of the present invention may exist in solvated, for example hydrated, as well as unsolvated forms, and the present invention encompasses all such forms.

Typically, but not absolutely, the salts of the present invention are pharmaceutically acceptable salts. Salts encompassed within the term “pharmaceutically acceptable salts” refer to non-toxic salts of the compounds of this invention.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

### II. General Synthetic Methods

For ease of reference, the following numbering systems may be used to refer to particular scaffolds of the present invention or scaffolds used as intermediates in the synthesis thereof and such numbering is believed consistent with convention:

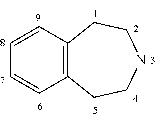

2,3,4,5-tetrahydro-1H-benzo[d]azepine or 2,3,4,5-tetrahydro-1H-3-benzazepine

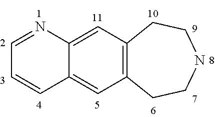

7,8,9,10-tetrahydro-6H-azepino[4,5-g]quinoline

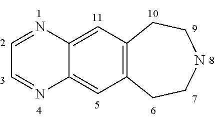

7,8,9,10-tetrahydro-6H-azepino[4,5-g]quinoxaline

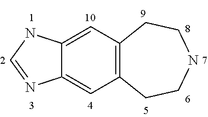

1,5,6,7,8,9-hexahydroimidazo[4,5-h][3]benzazepine

Among compounds of the present invention, 7,8,9,10-tetrahydro-6H-azepino[4,5-g]quinoxaline, 7,8,9,10-tetrahydro-6H-azepino[4,5-g]quinoline, and 1,5,6,7,8,9-hexahydroimidazo[4,5-h][3]benzazepine, and derivatives thereof, can be prepared from commercially available 2,3,4,5-tetrahydro-1H-benzo[d]azepine (also known as 2,3,4,5-tetrahydro-1H-3-benzazepine) using modifications of procedures found in using modifications of procedures found in U.S. Pat. No. 6,605,610, incorporated by reference with regard to synthetic procedures described in column 14, line 43 to column 16, line 35; column 17, lines 36 to 65; schemes 2 and 5; and the synthetic examples.

As demonstrated in the Examples, 2,3,4,5-tetrahydro-1H-benzo[d]azepine (available from Ramidus AB) can first be converted to its trifluoroacetamide derivative. The resulting material, 3-trifluoroacetyl-2,3,4,5-tetrahydro-1H-benzo[d]azepine (also known as 3-trifluoroacetyl-2,3,4,5-tetrahydro-1H-3-benzazepine), can be nitrated using a mixture of fuming nitric acid and triflic acid. The conditions of the nitration reaction can be varied to provide either the mono-nitro or the di-nitro product, either of which is useful in the production of compounds of the present invention. The nitration products, 7-nitro-3-trifluoroacetyl-2,3,4,5-tetrahydro-1H-benzo[d]azepine and 7,8-dinitro-3-trifluoroacetyl-2,3,4,5-tetrahydro-1H-benzo[d]azepine, are then reduced (for instance by palladium-catalyzed hydrogenation) to the corresponding mono- and di-amines, 7-amino-3-trifluoroacetyl-2,3,4,5-tetrahydro-1H-benzo[d]azepine and 7,8-diamino-3-trifluoroacetyl-2,3,4,5-tetrahydro-1H-benzo[d]azepine. The 7-amino-3-trifluoroacetyl-2,3,4,5-tetrahydro-1H-benzo[d]azepine can be converted, via combinations of chemical transformation, into 7,8,9,10-tetrahydro-6H-azepino[4,5-g]quinoline and derivatives thereof, as outlined in Scheme 1. The 7,8-diamino-3-trifluoroacetyl-2,3,4,5-tetrahydro-1H-benzo[d]azepine can be converted, via combinations of chemical transformation, into 7,8,9,10-tetrahydro-6H-azepino[4,5-g]quinoxaline and derivatives thereof, as outlined in Scheme 2. The 7,8-diamino-3-trifluoroacetyl-2,3,4,5-tetrahydro-1H-benzo[d]azepine can also be converted, via combinations of chemical transformation, into 1,5,6,7,8,9-hexahydroimidazo[4,5-h][3]benzazepine and derivatives thereof.

As detailed in the Examples and outlined in Scheme 1, the compound 7,8,9,10-tetrahydro-6H-azepino[4,5-g]quinoline can be accessed by the condensation of 7-amino-3-trifluoroacetyl-2,3,4,5-tetrahydro-1H-benzo[d]azepine with appropriate electrophilic reagents (e.g., glycerol in the presence of sulfuric acid and catalytic iodine). Various 7,8,9,10-tetrahydro-6H-azepino[4,5-g]quinoline derivatives can be produced from 8-trifluoroacetyl-7,8,9,10-tetrahydro-6H-azepino[4,5-g]quinolin-2(1H)-one, which is the condensation product of 7-amino-3-trifluoroacetyl-2,3,4,5-tetrahydro-1H-benzo[d]azepine with 3,3-diethoxypropanoic acid in the presence of dicyclohexylcarbodimide. Many of these synthetic transformations, by which 7,8,9,10-tetrahydro-6H-azepino[4,5-g]quinoline and its derivatives are made, are well-known to those of skill in the art of synthetic chemistry.

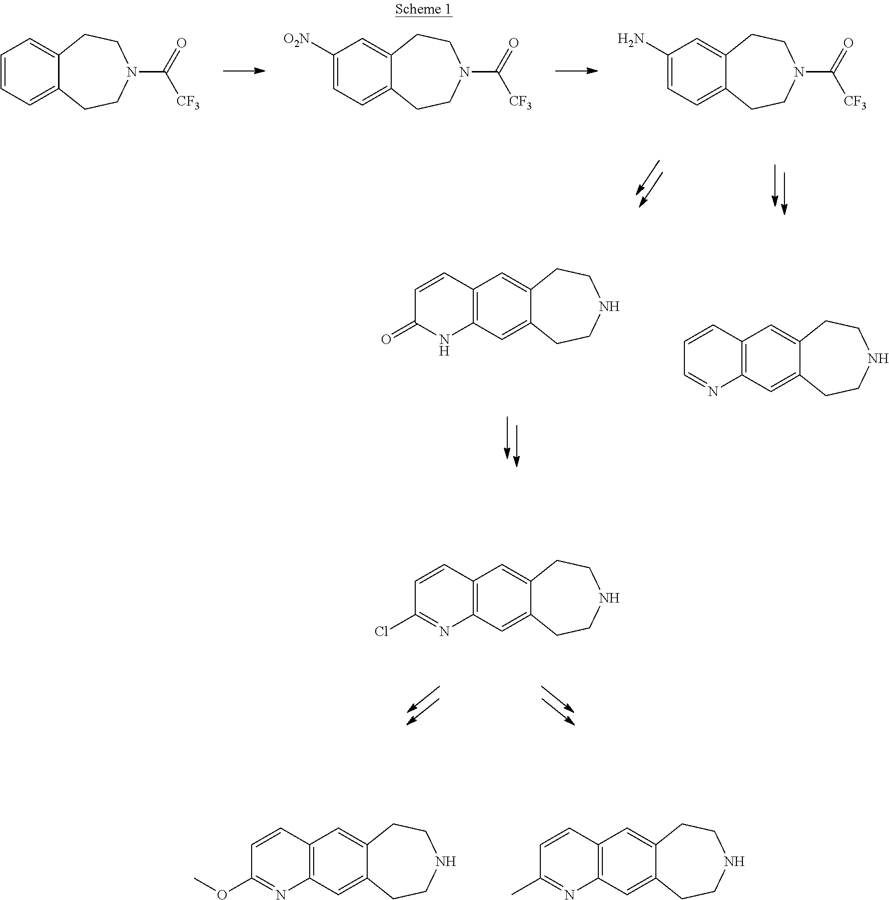

As detailed in the Examples and outlined in Scheme 2, the compound 7,8,9,10-tetrahydro-6H-azepino[4,5-g]quinoxaline can be accessed by condensation of 7,8-diamino-3-trifluoroacetyl-2,3,4,5-tetrahydro-1H-benzo[d]azepine with glyoxal, followed by removal of the trifluoroacetyl protecting group. Other reagents, such as p-dioxane-2,3-diol, can also be used to transform an appropriately protected 7,8-diamino-2,3,4,5-tetrahydro-1 H-benzo[d]azepine (also known as 7,8-diamino-1,2,4,5-tetrahydro-3H-3-benzazepine) into the corresponding protected 7,8,9,10-tetrahydro-6H-azepino[4,5-g]quinoxaline. The analogous condensations using 2-oxopropanal give, following removal of the protecting group, 2-methyl-7,8,9,10-tetrahydro-6H-azepino[4,5-g]quinoxaline. Various 7,8,9,10-tetrahydro-6H-azepino[4,5-g]quinoxaline derivatives can be produced from 8-trifluoroacetyl-7,8,9,10-tetrahydro-6H-azepino[4,5-g]quinoxalin-2(1H)-one, which is the condensation product of 7,8-diamino-3-trifluoroacetyl-2,3,4,5-tetrahydro-1H-benzo[d]azepine with ethyl glyoxylate. Many of these synthetic transformations, by which 7,8,9,10-tetrahydro-6H-azepino[4,5-g]quinoxaline and its derivatives are made, are well-known to those of skill in the art of synthetic chemistry.

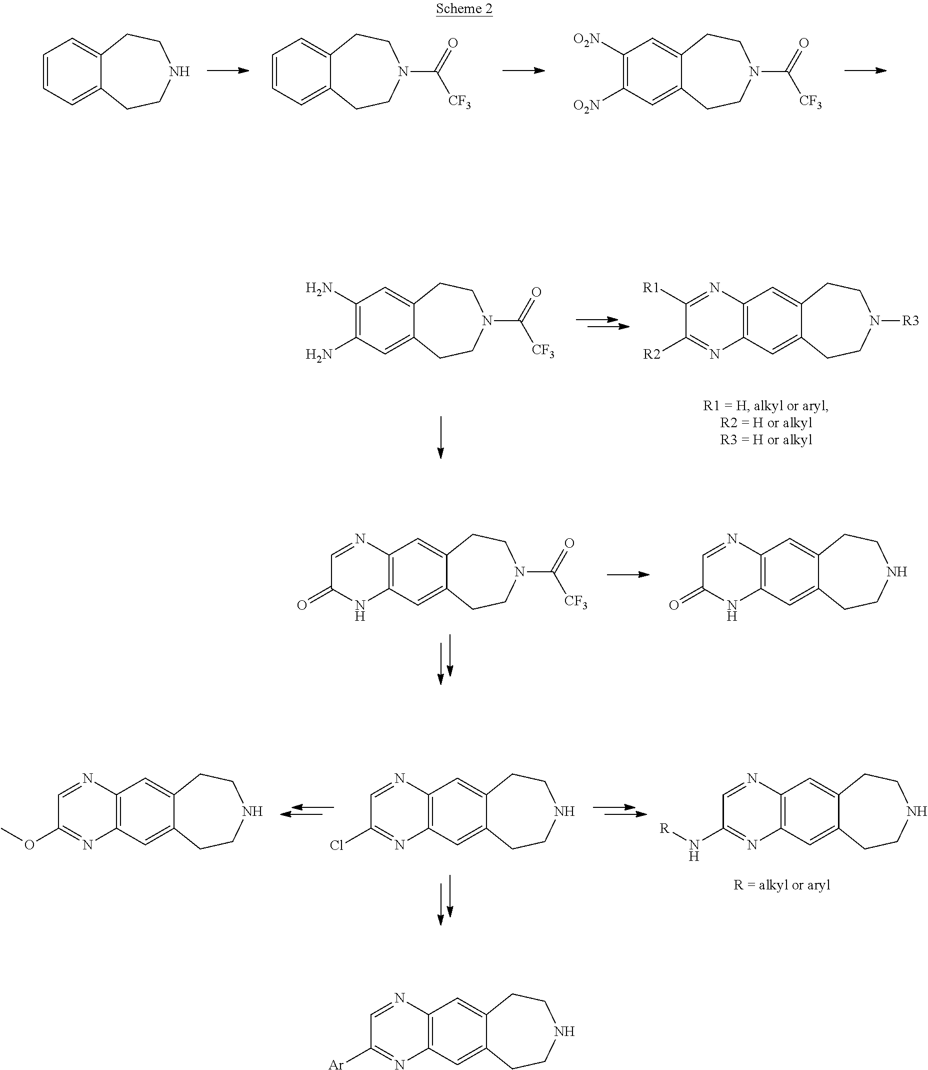

### IV. Pharmaceutical Compositions

Although it is possible to administer the compound of the present invention in the form of a bulk active chemical, it is preferred to administer the compound in the form of a pharmaceutical composition or formulation. Thus, one aspect the present invention includes pharmaceutical compositions comprising the compound of the present invention and one or more pharmaceutically acceptable carriers, diluents, or excipients. Another aspect of the invention provides a process for the preparation of a pharmaceutical composition including admixing the compound of the present invention with one or more pharmaceutically acceptable carriers, diluents or excipients.

The manner in which the compound of the present invention is administered can vary. The compound of the present invention is preferably administered orally. Preferred pharmaceutical compositions for oral administration include tablets, capsules, caplets, syrups, solutions, and suspensions. The pharmaceutical compositions of the present invention may be provided in modified release dosage forms such as time-release tablet and capsule formulations.

The pharmaceutical compositions can also be administered via injection, namely, intravenously, intramuscularly, subcutaneously, intraperitoneally, intraarterially, intrathecally, and intracerebroventricularly. Intravenous administration is a preferred method of injection. Suitable carriers for injection are well known to those of skill in the art and include 5% dextrose solutions, saline, and phosphate buffered saline. The formulations may also be administered using other means, for example, rectal administration. Formulations useful for rectal administration, such as suppositories, are well known to those of skill in the art. The compounds can also be administered by inhalation, for example, in the form of an aerosol; topically, such as, in lotion form; transdermally, such as, using a transdermal patch (for example, by using technology that is commercially available from Novartis and Alza Corporation), by powder injection, or by buccal, sublingual, or intranasal absorption.

Pharmaceutical compositions may be formulated in unit dose form, or in multiple or subunit doses

The administration of the pharmaceutical compositions described herein can be intermittent, or at a gradual, continuous, constant or controlled rate. The pharmaceutical compositions may be administered to a warm-blooded animal, for example, a mammal such as a mouse, rat, cat, rabbit, dog, pig, cow, or monkey; but advantageously is administered to a human being. In addition, the time of day and the number of times per day that the pharmaceutical composition is administered can vary.

The compound of the present invention may be used in the treatment of a variety of disorders and conditions and, as such, may be used in combination with a variety of other suitable therapeutic agents useful in the treatment or prophylaxis of those disorders or conditions. Thus, one embodiment of the present invention includes the administration of the compound of the present invention in combination with other therapeutic compounds. For example, the compound of the present invention can be used in combination with other NNR ligands (such as varenicline), antioxidants (such as free radical scavenging agents), antibacterial agents (such as penicillin antibiotics), antiviral agents (such as nucleoside analogs, like zidovudine and acyclovir), anticoagulants (such as warfarin), anti-inflammatory agents (such as NSAIDs), anti-pyretics, analgesics, anesthetics (such as used in surgery), acetylcholinesterase inhibitors (such as donepezil and galantamine), antipsychotics (such as haloperidol, clozapine, olanzapine, and quetiapine), immuno-suppressants (such as cyclosporin and methotrexate), neuroprotective agents, steroids (such as steroid hormones), corticosteroids (such as dexamethasone, predisone, and hydrocortisone), vitamins, minerals, nutraceuticals, anti-depressants (such as imipramine, fluoxetine, paroxetine, escitalopram, sertraline, venlafaxine, and duloxetine), anxiolytics (such as alprazolam and buspirone), anticonvulsants (such as phenytoin and gabapentin), vasodilators (such as prazosin and sildenafil), mood stabilizers (such as valproate and aripiprazole), anti-cancer drugs (such as anti-proliferatives), antihypertensive agents (such as atenolol, clonidine, amlopidine, verapamil, and olmesartan), laxatives, stool softeners, diuretics (such as furosemide), anti-spasmotics (such as dicyclomine), anti-dyskinetic agents, and anti-ulcer medications (such as esomeprazole). Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the present invention with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second. Such sequential administration may be close in time or remote in time.

Another aspect of the present invention includes combination therapy comprising administering to the subject a therapeutically or prophylactically effective amount of the compound of the present invention and one or more other therapy including chemotherapy, radiation therapy, gene therapy, or immunotherapy.

### IV. Method of Using Pharmaceutical Compositions

The compounds of the present invention can be used for the prevention or treatment of various conditions or disorders for which other types of nicotinic compounds have been proposed or are shown to be useful as therapeutics, such as CNS disorders, inflammation, inflammatory response associated with bacterial and/or viral infection, pain, metabolic syndrome, autoimmune disorders, addictions, obesity or other disorders described in further detail herein. This compound can also be used as a diagnostic agent in receptor binding studies (in vitro and in vivo). Such therapeutic and other teachings are described, for example, in references previously listed herein, including Williams et al., Drug News Perspec. 7(4): 205 (1994), Arneric et al., CNS Drug Rev. 1(1): 1-26 (1995), Arneric et al., Exp. Opin. Invest. Drugs 5(1): 79-100 (1996), Bencherif et al., J. Pharmacol. Exp. Ther. 279: 1413 (1996), Lippiello et al., J. Pharmacol. Exp. Ther. 279: 1422 (1996), Damaj et al., J. Pharmacoi. Exp. Ther. 291: 390 (1999); Chiari et al., Anesthesiology 91: 1447 (1999), Lavand'homme and Eisenbach, Anesthesiology 91: 1455 (1999), Holladay et al., J. Med. Chem. 40(28): 4169-94 (1997), Bannon et al., Science 279: 77 (1998), PCT WO 94/08992, PCT WO 96/31475, PCT WO 96/40682, and U.S. Pat. No. 5,583,140 to Bencherif et al., U.S. Pat. No. 5,597,919 to Dull et al., U.S. Pat. No. 5,604,231 to Smith et al. and U.S. Pat. No. 5,852,041 to Cosford et al.

### CNS Disorders

The compounds and their pharmaceutical compositions are useful in the treatment or prevention of a variety of CNS disorders, including neurodegenerative disorders, neuropsychiatric disorders, neurologic disorders, and addictions. The compounds and their pharmaceutical compositions can be used to treat or prevent cognitive deficits and dysfunctions, age-related and otherwise; attentional disorders and dementias, including those due to infectious agents or metabolic disturbances; to provide neuroprotection; to treat convulsions and multiple cerebral infarcts; to treat mood disorders, compulsions and addictive behaviors; to provide analgesia; to control inflammation, such as mediated by cytokines and nuclear factor kappa B; to treat inflammatory disorders; to provide pain relief; and to treat infections, as anti-infectious agents for treating bacterial, fungal, and viral infections. Among the disorders, diseases and conditions that the compounds and pharmaceutical compositions of the present invention can be used to treat or prevent are: age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, central ischemia, peripheral ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, tardive dyskinesia, spastic dystonia, hyperkinesia, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), and chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, conduct disorder, oppositional defiant disorder, Tourette's syndrome, autism, drug and alcohol addiction, tobacco addiction, compulsive overeating and sexual dysfunction.

Cognitive impairments or dysfunctions may be associated with psychiatric disorders or conditions, such as schizophrenia and other psychotic disorders, including but not limited to psychotic disorder, schizophreniform disorder, schizoaffective disorder, delusional disorder, brief psychotic disorder, shared psychotic disorder, and psychotic disorders due to a general medical conditions, dementias and other cognitive disorders, including but not limited to mild cognitive impairment, pre-senile dementia, Alzheimer's disease, senile dementia, dementia of the Alzheimer's type, age-related memory impairment, Lewy body dementia, vascular dementia, AIDS dementia complex, dyslexia, Parkinsonism including Parkinson's disease, cognitive impairment and dementia of Parkinson's Disease, cognitive impairment of multiple sclerosis, cognitive impairment caused by traumatic brain injury, dementias due to other general medical conditions, anxiety disorders, including but not limited to panic disorder without agoraphobia, panic disorder with agoraphobia, agoraphobia without history of panic disorder, specific phobia, social phobia, obsessive-compulsive disorder, post-traumatic stress disorder, acute stress disorder, generalized anxiety disorder and generalized anxiety disorder due to a general medical condition, mood disorders, including but not limited to major depressive disorder, dysthymic disorder, bipolar depression, bipolar mania, bipolar I disorder, depression associated with manic, depressive or mixed episodes, bipolar II disorder, cyclothymic disorder, and mood disorders due to general medical conditions, sleep disorders, including but not limited to dyssomnia disorders, primary insomnia, primary hypersomnia, narcolepsy, parasomnia disorders, nightmare disorder, sleep terror disorder and sleepwalking disorder, mental retardation, learning disorders, motor skills disorders, communication disorders, pervasive developmental disorders, attention-deficit and disruptive behavior disorders, attention deficit disorder, attention deficit hyperactivity disorder, feeding and eating disorders of infancy, childhood, or adults, tic disorders, elimination disorders, substance-related disorders, including but not limited to substance dependence, substance abuse, substance intoxication, substance withdrawal, alcohol-related disorders, amphetamine or amphetamine-like-related disorders, caffeine-related disorders, cannabis-related disorders, cocaine-related disorders, hallucinogen-related disorders, inhalant-related disorders, nicotine-related disorders, opioid-related disorders, phencyclidine or phencyclidine-like-related disorders, and sedative-, hypnotic- or anxiolytic-related disorders, personality disorders, including but not limited to obsessive-compulsive personality disorder and impulse-control disorders.

Cognitive performance may be assessed with a validated cognitive scale, such as, for example, the cognitive subscale of the Alzheimer's

Disease Assessment Scale (ADAS-cog). One measure of the effectiveness of the compounds of the present invention in improving cognition may include measuring a patient's degree of change according to such a scale.

Regarding compulsions and addictive behaviors, the compounds of the present invention may be used as a therapy for nicotine addiction and for other brain-reward disorders, such as substance abuse including alcohol addiction, illicit and prescription drug addiction, eating disorders, including obesity, and behavioral addictions, such as gambling, or other similar behavioral manifestations of addiction.

The above conditions and disorders are discussed in further detail, for example, in the American Psychiatric Association: Diagnostic and Statistical Manual of Mental Disorders, Fourth Edition, Text Revision, Washington, D.C., American Psychiatric Association, 2000. This Manual may also be referred to for greater detail on the symptoms and diagnostic features associated with substance use, abuse, and dependence.

Preferably, the treatment or prevention of diseases, disorders and conditions occurs without appreciable adverse side effects, including, for example, significant increases in blood pressure and heart rate, significant negative effects upon the gastro-intestinal tract, and significant effects upon skeletal muscle.

The compounds of the present invention, when employed in effective amounts, are believed to modulate the activity of the α4β2 NNR subtype without appreciable interaction with the nicotinic subtypes that characterize the human ganglia, as demonstrated by a lack of the ability to elicit nicotinic function in adrenal chromaffin tissue, or skeletal muscle, further demonstrated by a lack of the ability to elicit nicotinic function in cell preparations expressing muscle-type nicotinic receptors. Thus, these compounds are believed capable of treating or preventing diseases, disorders and conditions without eliciting significant side effects associated activity at ganglionic and neuromuscular sites. Thus, administration of the compounds is believed to provide a therapeutic window in which treatment of certain diseases, disorders and conditions is provided, and certain side effects are avoided. That is, an effective dose of the compound is believed sufficient to provide the desired effects upon the disease, disorder or condition, but is believed insufficient, namely is not at a high enough level, to provide undesirable side effects.

Thus, the present invention provides the use of a compound of the present invention, or a pharmaceutically acceptable salt thereof, for use in therapy, such as a therapy described above.

In yet another aspect the present invention provides the use of a compound of the present invention, or a pharmaceutically acceptable salt thereof, in the manufacture of a medicament for use in the treatment of a CNS disorder, such as a disorder, disease or condition described hereinabove.

### Inflammation

The nervous system, primarily through the vagus nerve, is known to regulate the magnitude of the innate immune response by inhibiting the release of macrophage tumor necrosis factor (TNF). This physiological mechanism is known as the “cholinergic anti-inflammatory pathway” (see, for example, Tracey, “The Inflammatory Reflex,” Nature 420: 853-9 (2002)). Excessive inflammation and tumor necrosis factor synthesis cause morbidity and even mortality in a variety of diseases. These diseases include, but are not limited to, endotoxemia, rheumatoid arthritis, osteoarthritis, psoriasis, asthma, atherosclerosis, idiopathic pulmonary fibrosis, and inflammatory bowel disease.

Inflammatory conditions that can be treated or prevented by administering the compounds described herein include, but are not limited to, chronic and acute inflammation, psoriasis, endotoxemia, gout, acute pseudogout, acute gouty arthritis, arthritis, rheumatoid arthritis, osteoarthritis, allograft rejection, chronic transplant rejection, asthma, atherosclerosis, mononuclear-phagocyte dependent lung injury, idiopathic pulmonary fibrosis, atopic dermatitis, chronic obstructive pulmonary disease, adult respiratory distress syndrome, acute chest syndrome in sickle cell disease, inflammatory bowel disease, irritable bowel syndrome, Crohn's disease, ulcers, ulcerative colitis, acute cholangitis, aphthous stomatitis, cachexia, pouchitis, glomerulonephritis, lupus nephritis, thrombosis, and graft vs. host reaction.

Inflammatory Response Associated with Bacterial and/or Viral Infection

Many bacterial and/or viral infections are associated with side effects brought on by the formation of toxins, and the body's natural response to the bacteria or virus and/or the toxins. As discussed above, the body's response to infection often involves generating a significant amount of TNF and/or other cytokines. The over-expression of these cytokines can result in significant injury, such as septic shock (when the bacteria is sepsis), endotoxic shock, urosepsis, viral pneumonitis and toxic shock syndrome.

Cytokine expression is mediated by NNRs, and can be inhibited by administering agonists or partial agonists of these receptors. Those compounds described herein that are agonists or partial agonists of these receptors can therefore be used to minimize the inflammatory response associated with bacterial infection, as well as viral and fungal infections. Examples of such bacterial infections include anthrax, botulism, and sepsis. Some of these compounds may also have antimicrobial properties.

These compounds can also be used as adjunct therapy in combination with existing therapies to manage bacterial, viral and fungal infections, such as antibiotics, antivirals and antifungals. Antitoxins can also be used to bind to toxins produced by the infectious agents and allow the bound toxins to pass through the body without generating an inflammatory response. Examples of antitoxins are disclosed, for example, in U.S. Pat. No. 6,310,043 to Bundle et al. Other agents effective against bacterial and other toxins can be effective and their therapeutic effect can be complemented by co-administration with the compounds described herein.

### Pain

The compounds can be administered to treat and/or prevent pain, including acute, neurologic, inflammatory, neuropathic and chronic pain. The compounds can be used in conjunction with opiates to minimize the likelihood of opiate addiction (e.g., morphine sparing therapy). The analgesic activity of compounds described herein can be demonstrated in models of persistent inflammatory pain and of neuropathic pain, performed as described in U.S. Published Patent Application No. 20010056084 Al (Allgeier et al.) (e.g., mechanical hyperalgesia in the complete Freund's adjuvant rat model of inflammatory pain and mechanical hyperalgesia in the mouse partial sciatic nerve ligation model of neuropathic pain).

The analgesic effect is suitable for treating pain of various genesis or etiology, in particular in treating inflammatory pain and associated hyperalgesia, neuropathic pain and associated hyperalgesia, chronic pain (e.g., severe chronic pain, post-operative pain and pain associated with various conditions including cancer, angina, renal or biliary colic, menstruation, migraine, and gout). Inflammatory pain may be of diverse genesis, including arthritis and rheumatoid disease, teno-synovitis and vasculitis. Neuropathic pain includes trigeminal or herpetic neuralgia, neuropathies such as diabetic neuropathy pain, causalgia, low back pain and deafferentation syndromes such as brachial plexus avulsion.

### Neovascularization

The α7 NNR is associated with neovascularization. Inhibition of neovascularization, for example, by administering antagonists (or at certain dosages, partial agonists) of the α7 NNR can treat or prevent conditions characterized by undesirable neovascularization or angiogenesis. Such conditions can include those characterized by inflammatory angiogenesis and/or ischemia-induced angiogenesis. Neovascularization associated with tumor growth can also be inhibited by administering those compounds described herein that function as antagonists or partial agonists of α7 NNR.

Specific antagonism of α7 NNR-specific activity reduces the angiogenic response to inflammation, ischemia, and neoplasia. Guidance regarding appropriate animal model systems for evaluating the compounds described herein can be found, for example, in Heeschen, C. et aL, “A novel angiogenic pathway mediated by non-neuronal nicotinic acetylcholine receptors,” J. Clin. Invest. 110(4):527-36 (2002).

Representative tumor types that can be treated using the compounds described herein include NSCLC, ovarian cancer, pancreatic cancer, breast carcinoma, colon carcinoma, rectum carcinoma, lung carcinoma, oropharynx carcinoma, hypopharynx carcinoma, esophagus carcinoma, stomach carcinoma, pancreas carcinoma, liver carcinoma, gallbladder carcinoma, bile duct carcinoma, small intestine carcinoma, urinary tract carcinoma, kidney carcinoma, bladder carcinoma, urothelium carcinoma, female genital tract carcinoma, cervix carcinoma, uterus carcinoma, ovarian carcinoma, choriocarcinoma, gestational trophoblastic disease, male genital tract carcinoma, prostate carcinoma, seminal vesicles carcinoma, testes carcinoma, germ cell tumors, endocrine gland carcinoma, thyroid carcinoma, adrenal carcinoma, pituitary gland carcinoma, skin carcinoma, hemangiomas, melanomas, sarcomas, bone and soft tissue sarcoma, Kaposi's sarcoma, tumors of the brain, tumors of the nerves, tumors of the eyes, tumors of the meninges, astrocytomas, gliomas, glioblastomas, retinoblastomas, neuromas, neuroblastomas, Schwannomas, meningiomas, solid tumors arising from hematopoietic malignancies (such as leukemias, chloromas, plasmacytomas and the plaques and tumors of mycosis fungoides and cutaneous T-cell lymphoma/leukemia), and solid tumors arising from lymphomas.

The compounds can also be administered in conjunction with other forms of anti-cancer treatment, including co-administration with antineoplastic antitumor agents such as cis-platin, adriamycin, daunomycin, and the like, and/or anti-VEGF (vascular endothelial growth factor) agents, as such are known in the art.

The compounds can be administered in such a manner that they are targeted to the tumor site. For example, the compounds can be administered in microspheres, microparticles or liposomes conjugated to various antibodies that direct the microparticles to the tumor. Additionally, the compounds can be present in microspheres, microparticles or liposomes that are appropriately sized to pass through the arteries and veins, but lodge in capillary beds surrounding tumors and administer the compounds locally to the tumor. Such drug delivery devices are known in the art.

### Other Disorders

In addition to treating CNS disorders, inflammation, and neovascularization, and pain, the compounds of the present invention can be also used to prevent or treat certain other conditions, diseases, and disorders in which NNRs play a role. Examples include autoimmune disorders such as lupus, disorders associated with cytokine release, cachexia secondary to infection (e.g., as occurs in AIDS, AIDS related complex and neoplasia), obesity, pemphitis, urinary incontinence, overactive bladder, diarrhea, constipation, retinal diseases, infectious diseases, myasthenia, Eaton-Lambert syndrome, hypertension, preeclampsia, osteoporosis, vasoconstriction, vasodilatation, cardiac arrhythmias, type I diabetes, type II diabetes, bulimia, anorexia and sexual dysfunction, as well as those indications set forth in published PCT application WO 98/25619. The compounds of this invention can also be administered to treat convulsions such as those that are symptomatic of epilepsy, and to treat conditions such as syphillis and Creutzfeld-Jakob disease.

### Diagnostic Uses

The compounds can be used in diagnostic compositions, such as probes, particularly when they are modified to include appropriate labels. The probes can be used, for example, to determine the relative number and/or function of specific receptors, particularly the α4β2 and α7 receptor subtypes. For this purpose the compounds of the present invention most preferably are labeled with a radioactive isotopic moiety such as ¹¹C, ¹⁸F, ⁷⁶Br, ¹²³I or ¹²⁵I.

The administered compounds can be detected using known detection methods appropriate for the label used. Examples of detection methods include position emission topography (PET) and single-photon emission computed tomography (SPECT). The radiolabels described above are useful in PET (e.g., ¹¹C, ¹⁸F or ⁷⁶Br) and SPECT (e.g., ¹²³I) imaging, with half-lives of about 20.4 minutes for ¹¹C, about 109 minutes for ¹⁸F, about 13 hours for ¹²³I, and about 16 hours for ⁷⁶Br. A high specific activity is desired to visualize the selected receptor subtypes at non-saturating concentrations. The administered doses typically are below the toxic range and provide high contrast images. The compounds are expected to be capable of administration in non-toxic levels. Determination of dose is carried out in a manner known to one skilled in the art of radiolabel imaging. See, for example, U.S. Pat. No. 5,969,144 to London et al.

The compounds can be administered using known techniques. See, for example, U.S. Pat. No. 5,969,144 to London et al., as noted. The compounds can be administered in formulation compositions that incorporate other ingredients, such as those types of ingredients that are useful in formulating a diagnostic composition. Compounds useful in accordance with carrying out the present invention most preferably are employed in forms of high purity. See, U.S. Pat. No. 5,853,696 to Elmalch et al.

After the compounds are administered to a subject (e.g., a human subject), the presence of that compound within the subject can be imaged and quantified by appropriate techniques in order to indicate the presence, quantity, and functionality of selected NNR subtypes. In addition to humans, the compounds can also be administered to animals, such as mice, rats, dogs, and monkeys. SPECT and PET imaging can be carried out using any appropriate technique and apparatus. See Villemagne et al., In: Arneric et al. (Eds.) Neuronal Nicotinic Receptors: Pharmacology and Therapeutic Opportunities, 235-250 (1998) and U.S. Pat. No. 5,853,696 to Elmalch et al.

The radiolabeled compounds bind with high affinity to selective NNR subtypes (e.g., α4β2, α7) and preferably exhibit negligible non-specific binding to other nicotinic cholinergic receptor subtypes (e.g., those receptor subtypes associated with muscle and ganglia). As such, the compounds can be used as agents for noninvasive imaging of nicotinic cholinergic receptor subtypes within the body of a subject, particularly within the brain for diagnosis associated with a variety of CNS diseases and disorders.

In one aspect, the diagnostic compositions can be used in a method to diagnose disease in a subject, such as a human patient. The method involves administering to that patient a detectably labeled compound as described herein, and detecting the binding of that compound to selected NNR subtypes (e.g., α4β2 and α7 receptor subtypes). Those skilled in the art of using diagnostic tools, such as PET and SPECT, can use the radiolabeled compounds described herein to diagnose a wide variety of conditions and disorders, including conditions and disorders associated with dysfunction of the central and autonomic nervous systems. Such disorders include a wide variety of CNS diseases and disorders, including Alzheimer's disease, Parkinson's disease, and schizophrenia. These and other representative diseases and disorders that can be evaluated include those that are set forth in U.S. Pat. No. 5,952,339 to Bencherif et al.

In another aspect, the diagnostic compositions can be used in a method to monitor selective nicotinic receptor subtypes of a subject, such as a human patient. The method involves administering a detectably labeled compound as described herein to that patient and detecting the binding of that compound to selected nicotinic receptor subtypes namely, the α4β2 and α7 receptor subtypes.

### Receptor Binding

The compounds of this invention can be used as reference ligands in binding assays for compounds which bind to NNR subtypes, particularly the α4β2 and α7 receptor subtypes. For this purpose the compounds of this invention are preferably labeled with a radioactive isotopic moiety such as ³H, or ¹⁴C. Examples of such binding assays are described in detail below.

### V. Synthetic Examples

### Example 1

Example 1 details the synthesis of 7,8,9,10-tetrahydro-6H-azepino[4,5-g]quinoxaline.

3-Trifluoroacetyl-2,3,4,5-tetrahydro-1H-benzo[d]azepine
Trifluoroacetic anhydride (33.92 g, 161.5 mmol) was added drop-wise under nitrogen atmosphere to a cooled (0° C.) solution of 2,3,4,5-tetrahydro-1H-benzo[d]azepine (19.0 g, 129 mmol) and pyridine (15.3 g, 193 mmol) in anhydrous dichloromethane (650 mL). The reaction mixture was warmed to ambient temperature, stirred for 16 h and poured into water (200 mL). The mixture was shaken well, and the organic layer was separated, washed with 0.5 M hydrochloric acid (200 mL) and dried over anhydrous sodium sulfate. The sodium sulfate was removed by gravity filtration, and the filtrate was concentrated by rotary evaporation. The residue was purified by flash chromatography, using an ethyl acetate in hexanes step-wise gradient (0 to 100% ethyl acetate). Concentration of selected fractions gave 30.5 g (97% yield) of 3-trifluoroacetyl-2,3,4,5-tetrahydro-1H-benzo[d]azepine. ¹H NMR (CDCl₃, 300 MHz): [symbol] 7.22-7.10 (m, 4H), 3.8-3.65 (m, 4H), 3.0 (m, 4H).
7,8-Dinitro-3-trifluoroacetyl-2,3,4,5-tetrahydro-1H-benzo[d]azepine
Fuming (90%) nitric acid (2.68 g, 37.8 mmol) was added drop-wise to a solution of trifluoromethanesulfonic acid (11.35 g, 75.67 mmol) in dichloromethane (80 mL) at 0° C. and stirred for 10 min. A solution of 3-trifluoroacetyl-2,3,4,5-tetrahydro-1H-benzo[d]azepine (4.00 g, 16.5 mmol) in dichloromethane (10 mL) was then added drop-wise, and the mixture was stirred 1 h at 0° C. The reaction mixture was warmed to ambient temperature, stirred for 16 h, poured into water (50 mL) and extracted with dichloromethane (2×50 mL). The combined organic extracts were washed with water, dried (anhydrous sodium sulfate) and concentrated. The residue was purified by flash chromatography, eluting with an ethyl acetate in hexanes gradient (0 to 100% ethyl acetate). Concentration of selected fractions gave a mixture of 7,8-dinitro-3-trifluoroacetyl-2,3,4,5-tetrahydro-1H-benzo[d]azepine and isomeric impurities (4.32 g, 78.9% yield). ¹H NMR (CDCl₃, 300 MHz): δ8.50 (m, 1H), 8.22 (m, 1H), 4.05-3.85 (m, 4H), 3.40-3.15 (m, 4H).
7,8-Diamino-3-trifluoroacetyl-2,3,4,5-tetrahydro-1H-benzo[d]azepine
Palladium hydroxide on carbon (300 mg of 20%, wet) was added, under an nitrogen atmosphere, to a solution of 7,8-dinitro-3-trifluoroacetyl-2,3,4,5-tetrahydro-1H-benzo[d]azepine (1.2 g, 3.6 mmol) in 1:1 ethyl acetate/methanol (30 mL). The mixture was subjected to hydrogenation at 50 psi for 16 h. The catalyst was removed by suction filtration, and the filtrate was concentrated by rotary evaporation, followed by high vacuum treatment, leaving 7,8-diamino-3-trifluoroacetyl-2,3,4,5-tetrahydro-1H-benzo[d]azepine (with isomeric impurities) (0.98 g, 99% yield). ¹H NMR (CD₃OD, 300 MHz): δ6.65 (m, 1H), 6.58 (m, 1H), 3.85-3.75 (m, 4H), 3.05-2.90 (m, 4H). MS (m/z): 274 (M+1).
8-Trifluoroacetyl-7,8,9,10-tetrahydro-6H-azepino[4,5-g]quinoxaline
Glyoxal (0.133 g of 40% aqueous) was added to a solution of 7,8-diamino-3-trifluoroacetyl-2,3,4,5-tetrahydro-1H-benzo[d]azepine (0.570 g, 2.08 mmol) in THF (20 mL). The reaction mixture was heated to 60° C. for 16 h. The volatiles were removed by rotary evaporation, and the residue was purified by preparative HPLC, using mixtures of acetonitrile and 0.05% aqueous trifluoroacetic acid as mobile phase. Concentration of selected fractions gave 8-trifluoroacetyl-7,8,9,10-tetrahydro-6H-azepino[4,5-g]quinoxaline (0.199 g, 32.5% yield). ¹H NMR (CDCl₃, 300 MHz): 8 8.80 (s, 2H), 7.88 (d, 2H), 3.88-3.78 (m, 4H), 3.25-3.19 (m, 4H). MS (m/z): 296 (M+1).
7,8,9,10-Tetrahydro-6H-azepino[4,5-g]quinoxaline
Potassium carbonate (1.20 g, 8.67 mmol) was added to a solution of 8-trifluoroacetyl-7,8,9,10-tetrahydro-6H-azepino[4,5-g]quinoxaline (1.28 g, 4.34 mmol) in methanol (40 mL), and the mixture was stirred at ambient temperature for 16 h. The solids were removed by suction filtration, and the filtrate was concentrated by rotary evaporation. The residue was purified by preparative HPLC, using mixtures of acetonitrile and 0.05% aqueous trifluoroacetic acid as mobile phase. Selected fractions were concentrated, dissolved in methanol (50 mL) and treated with pre-washed Amberlyst® A-26 (OH) resin (Dow Chemical) to obtain, after thorough evaporation of the solvent, 0.62 g of 7,8,9,10-tetrahydro-6H-azepino[4,5-g]quinoxaline (72% yield). ¹H NMR (CD₃OD, 300 MHz): 8 8.80 (s, 2H), 7.83 (s, 2H), 3.20 (m, 4H), 3.0 (m, 4H). MS (m/z): 200 (M+1).

### Example 2

Example 2 details the synthesis of various analogs of 7,8,9,10-tetrahydro-6H-azepino[4,5-g]quinoxaline.

2-Methyl-7,8,9,10-tetrahydro-6H-azepino[4,5-g]quinoxaline
7,8-Diamino-3-trifluoroacetyl-2,3,4,5-tetrahydro-1H-benzo[d]azepine (50 mg, 0.19 mmol) and 2-oxopropanal (15 mg, 0.21 mmol) were dissolved in 1:1 THF/water (1 mL) and heated at 80° C. for 4 h. The reaction mixture was cooled to ambient temperature, and the solvents were removed by evaporation. The residue was dissolved in methanol (1 mL) and treated with potassium carbonate (52 mg, 0.38 mmol) and stirred at ambient temperature for 3 h. The solids were removed by suction filtration, and the filtrate was concentrated. The residue was purified by preparative HPLC, using mixtures of acetonitrile and 0.05% aqueous trifluoroacetic acid as mobile phase. Selected fractions were concentrated to give 7.4 mg (12% yield) of 2-methyl-7,8,9,10-tetrahydro-6H-azepino[4,5-g]quinoxaline trifluoroactetate. MS (m/z): 214 (M+H).
7,8,9,10-tetrahydro-6H-azepino[4,5-g]quinoxalin-2(1H)-one
To a solution of 7,8-diamino-3-trifluoroacetyl-2,3,4,5-tetrahydro-1H-benzo[d]azepine (510 mg, 1.86 mmol) in absolute ethanol (20 mL) was added ethyl glyoxylate (50% in toluene) (0.57 mL, 2.8 mmol) dropwise with stirring. After refluxing for 2 h, the solution was cooled to ambient temperature. The solids were collected by suction filtration, rinsed with absolute ethanol and dried under vacuum to give 7,8,9,10-tetrahydro-1H-azepino[4,5-g]quinoxalin-2(6H)-one protected as its trifluoracetamide (0.36 g, 62% yield). MS (m/z): 312 (M+H). A sample of this material (28 mg, 90 μmol) was dissolved in methanol (1 mL) and treated with potassium carbonate (5.0 mg, 36 μmol). The mixture was stirred for 3 h at ambient temperature. The solids were removed by suction filtration, and the residue was purified by preparative HPLC, using mixtures of acetonitrile and 0.05% aqueous trifluoroacetic acid as mobile phase. Selected fractions were concentrated to give 7,8,9,10-tetrahydro-6H-azepino[4,5-g]quinoxalin-2(1H)-one trifluoroacetate (5.6 mg, 29% yield) as white solid. ¹H NMR (CD₃OD, 300 MHz): 8 8.16 (s, 1H), 7.70 (s, 1H), 7.18 (s, 1H), 3.4-3.18 (m, 8H). (MS m/z: 216 (M+H).
2-Chloro-7,8,9,10-tetrahydro-6H-azepino[4,5-g]quinoxaline
8-Trifluoroacetyl-7,8,9,10-tetrahydro-6H-azepino[4,5-g]quinoxalin-2(1H)-one (70 mg, 0.25 mmol) was dissolved in phosphoryl chloride (0.2 mL) and heated at 110° C. for 2 h. The reaction mixture was cooled to ambient temperature, and concentrated under vacuum. Solid sodium bicarbonate (100 mg, 0.94 mmol) was added to the residue, and the mixture was partitioned between ethyl acetate and water (5 mL each). The organic layer and two ethyl actetate extracts (3 ml each) of the aqueous layer were combined and dried over anhydrous sodium sulfate. Evaporation of the volatiles left 2-chloro-8-trifluoroacetyl-7,8,9,10-tetrahydro-6H-azepino[4,5-g]quinoxaline as brown solid. This material was dissolved in 1:1 THF/water (1 mL) and treated with 20 mg (0.14 mmol) of potassium carbonate. After stirring at ambient temperature for 48 h, the reaction was diluted with ether (5 mL), and the solids were removed by suction filtration. The filtrate was concentrated, and the residue purified by preparative HPLC, using mixtures of acetonitrile and 0.05% aqueous trifluoroacetic acid as mobile phase. Selected fractions were concentrated to give (5.8 mg, 10% yield) of 2-chloro-7,8,9,10-tetrahydro-6H-azepino[4,5-g]quinoxaline trifluoroacetate. ¹H NMR (CD₃OD, 300 MHz): δ8.82 (s, 1H), 8.00 (s, 1H), 7.85 (s, 1H), 3.4-3.1 (m, 8H). MS (m/z): 234 (M+H).
2-Methoxy-7,8,9,10-tetrahydro-6H-azepino[4,5-g]quinoxaline
2-Chloro-8-trifluoroacetyl-7,8,9,10-tetrahydro-6H-azepino[4,5-g]quinoxaline (50 mg, 0.17 mmol) was dissolved in methanol (1 mL) and treated with potassium carbonate (20 mg, 0.14 mmol). After stirring for 16 h at ambient temperature, the mixture was suction filtered to remove the solids, and the filtrate was concentrated. The residue was purified by preparative HPLC, using mixtures of acetonitrile and 0.05% aqueous trifluoroacetic acid as mobile phase. Concentration of selected fractions gave 14.8 mg (38% yield) of 2-methoxy-7,8,9,10-tetrahydro-6H-azepino[4,5-g]quinoxaline trifluoroacetate. ¹H NMR (CD₃OD, 300 MHz): δ8.36 (s, 1H), 7.76 (s, 1H), 7.66 (s, 1H), 4.0 (s, 3H), 3.3-3.2 (m, 8H). MS (m/z): 230 (M+H).
2-(Pyridin-3-yl)-7,8,9,10-tetrahydro-6H-azepino[4,5-g]quinoxaline
A mixture of 2-chloro-8-trifluoroacetyl-7,8,9,10-tetrahydro-6H-azepino[4,5-g]quinoxaline (75 mg, 0.23 mmol), pyridin-3-ylboronic acid (80 mg, 0.65 mmol), tetrakis(triphenylphosphine)palladium(0) (15 mg, 13 μmol) and sodium carbonate (150 mg, 1.41 mmol) in 95:5 ethanol/water (2 mL) was heated at reflux for 16 h. The reaction mixture was cooled to ambient temperature and diluted with ether (10 mL). The solids were removed by filtration, and the filtrate was concentrated. The residue was purified by preparative HPLC, using mixtures of acetonitrile and 0.05% aqueous trifluoroacetic acid as mobile phase. Selected fractions were concentrated to give 2-(pyridin-3-yl)-7,8,9,10-tetrahydro-6H-azepino[4,5-g]quinoxaline trifluoroacetate (38 mg, 67% yield) as syrup. ¹H NMR (CD₃OD, 300 MHz): δ9.53 (d, 1H), 9.41 (s, 1H), 9.17 (m, 1H), 8.76 (m, 1H), 8.00-7.87 (m, 3H), 3.3-3.12 (m, 8H). MS (m/z): 277.
2-(N-Methylamino)-7,8,9,10-tetrahydro-6H-azepino[4,5-g]quinoxaline
A mixture of 2-chloro-8-trifluoroacetyl-7,8,9,10-tetrahydro-6H-azepino[4,5-g]quinoxaline (75 mg, 0.23 mmol) and methylamine in THF (2 mL of 2.0 M) was refluxed for 10 h. The reaction was cooled to ambient temperature, and the volatiles were removed by rotary evaporation. The residue was dissolved in methanol (1 mL) and treated with potassium carbonate (10 mg, 72 μmol). The solids were removed by suction filtration, and the residue was purified by preparative HPLC, using mixtures of acetonitrile and 0.05% aqueous trifluoroacetic acid as mobile phase. Concentration of selected fractions gave 2-(N-methylamino)-7,8,9,10-tetrahydro-6H-azepino[4,5-g]quinoxaline trifluoroacetate (32 mg, 62% yield) as yellow solid. ¹H NMR (CD₃OD, 300 MHz): δ8.36 (s, 1H), 7.76 (s, 1H), 7.66 (s, 1H), 4.0 (s, 3H), 3.3-3.2 (m, 8H). MS (m/z): 229 (M+H).
8-Methyl-7,8,9,10-tetrahydro-6H-azepino[4,5-g]quinoxaline
To a solution of 7,8,9,10-tetrahydro-6H-azepino[4,5-g]quinoxaline (10 mg, 50 μmol) in methanol (1 mL) was added formaldehyde (37% solution 20 μL, 250 μmol) followed by sodiumtriacetoxy borohydride (31 mg, 150 μmol) at ambient temperature. After stirring for 4h, filtered off the solids and the filtrate purifed by preparative HPLC, using mixtures of acetonitrile and 0.05% aqueous trifluoroacetic acid as mobile phase to give 8-methyl-7,8,9,10-tetrahydro-6H-azepino[4,5-g]quinoxaline trifluoroacetate as dark brown solid (5.6 mg, 34% yield). ¹H NMR (CD₃OD, 300 MHz): δ8.87 (s, 2H), 7.98 (s, 2H), 3.85 (m, 2H), 3.56-3.38 (m, 4H), 3.30-3.17 (m, 2H), 3.0 (s, 3H)). MS (m/z): 214 (M+H).

### Example 3

Example 3 details the synthesis of 7,8,9,10-tetrahydro-6H-azepino[4,5-g]quinoline.

7-Nitro-3-trifluoroacetyl-2,3,4,5-tetrahydro-1H-benzo[d]azepine
Fuming (90%) nitric acid (1.03 g, 16.5 mmol) was added drop-wise to a solution of trifluoromethanesulfonic acid (4.93 g, 32.9 mmol) in dichloromethane (20 mL) at 0° C. and stirred for 10 min. The reaction flask was then cooled to −78° C., and a solution of 3-trifluoroacetyl-2,3,4,5-tetrahydro-1H-benzo[d]azepine (4.00 g, 16.5 mmol) in dichloromethane (10 mL) was added drop-wise. The mixture was stirred 30 min at −78° C., warmed to 0° C., stirred for 30 min, warmed to ambient temperature and stirred for 16 h. The reaction mixture was poured into water (20 mL) and extracted with dichloromethane (2×50 mL). The combined organic extracts were washed with water, dried (anhydrous sodium sulfate) and concentrated by rotary evaporation. The residue was purified by flash chromatography, eluting with a gradient of ethyl acetate in hexanes (0 to 100% ethyl acetate) to get 7-nitro-3 -trifluoroacetyl-2,3,4,5-tetrahydro-1H-benzo[d]azepine (2.90 g, 61.2%). ¹H NMR (CDCl₃, 300 MHz): 8 8.05 (m, 2H), 7.38 (m, 1H), 3.8 (m, 4H), 3.15 (m, 4H).
7-Amino-3-trifluoroacetyl-2,3,4,5-tetrahydro-1H-benzo[d]azepine
7-Nitro-3-trifluoroacetyl-2,3,4,5-tetrahydro-1H-benzo[d]azepine (1.92 g, 6.66 mmol) was dissolved in 1:1 ethyl acetate/methanol (50 mL), and 10% Pd on C (1.3 g) was added under a nitrogen atmosphere. The resulting mixture was shaken for 24 h under 50 psi of hydrogen. The mixture was suction filtered, and the filtrate was concentrated by rotary evaporation to give 1.52 g (88.9% yield) of 7-amino-3-trifluoroacetyl-2,3,4,5-tetrahydro-1H-benzo[d]azepine. MS (m/z): 259 (M+H).
7,8,9,10-Tetrahydro-6H-azepino[4,5-g]quinoline
A mixture of 7-amino-3-trifluoroacetyl-2,3,4,5-tetrahydro-1H-benzo[d]azepine (70 mg, 0. 27 mmol), glycerol (149 mg, 1.62 mmol), iodine (20 mg, 79 μmol) and sulfuric acid (317 mg, 3.20 mmol) was heated to 170° C. for 1 h. The reaction mixture was cooled to ambient temperature and diluted with chloroform (5 mL). Enough 10% aqueous sodium hydroxide was added to make the mixture basic. The mixture was shaken and the organic layer separated. The aqueous layer was extracted (2×5 mL) with chloroform. The combined organic extracts were dried over anhydrous sodium sulfate, filtered and concentrated. The residue was purified by preparative HPLC, giving 7,8,9,10-tetrahydro-6H-azepino[4,5-g]quinoline (2.9 mg, 3.5% yield). ¹H NMR (CD₃OD, 300 MHz): 8 8.92 (d, 1H), 8.64 (d, 1H), 8.00 (s, 2H), 7.75 (m, 1H), 3.34-3.20 (m, 8H). MS (m/z): 199 (M+H).

### Example 4

Example 4 details the synthesis of various analogs of 7,8,9,10-tetrahydro-6H-azepino[4,5-g]quinoline.

7,8,9,10-Tetrahydro-6H-azepino[4,5-g]quinolin-2(1H)-one
A mixture of 7-amino-3-trifluoroacetyl-2,3,4,5-tetrahydro-1H-benzo[d]azepine (258 mg, 1.00 mmol), 3,3-diethoxypropanoic acid (162 mg, 1.00 mmol), and dicyclohexylcarbodimide (206 mg, 1.00 mmol) in dichloromethane (1.5 mL) was stirred at ambient temperature for 12 h and then heated to 40° C. for 1 h. The solids were removed by filtration, and filtrate was concentrated. The residue was dissolved in trifluoroacetic acid (2 mL) and stirred for 2 h at ambient temperature. The volatiles were removed by rotary evaporation, and the residue was purified by preparative HPLC, mixtures of using acetonitrile and 0.05% aqueous trifluoroacetic acid as mobile phase. Selected fractions were concentrated, and the residue was dissolved in methanol (1 mL) and stirred with potassium carbonate (10 mg, 72 μmol) at ambient temperature for 3 h. The solids were removed by suction filtration, and the filtrate was concentrated and purified by HPLC, using mixtures of acetonitrile and 0.05% aqueous trifluoroacetic acid as mobile phase, to give 7,8,9,10-tetrahydro-6H-azepino[4,5-g]quinolin-2(1H)-one trifluoroacetate (9.9 mg, 5% yield) as white solid. ¹H NMR (CD₃OD, 300 MHz): δ7.90 (d, 1H), 7.55 (s, 1H), 7.20 (s, 1H), 6.58 (d, 1H), 3.34-3.18 (m, 8H). MS m/z: 215 (M+H).
2-Chloro-7,8,9,10-tetrahydro-6H-azepino[4,5-g]quinoline
A mixture of 8-trifluoroacetyl-7,8,9,10-tetrahydro-6H-azepino[4,5-g]quinolin-2(1H)-one (458 mg, 1.56 mmol) and phosphoryl chloride (359 mg, 2.34 mmol) was heated at 110° C. for 2 h. The reaction mixture was cooled to ambient temperature and concentrated under vacuum. The residue was neutralized with solid sodium bicarbonate and partitioned between ethyl acetate and water (25 mL each). The organic layer was separated, and the aqueous layer extracted with ethyl acetate (25 mL). The combined organic extracts were dried over anhydrous sodium sulfate and concentrated. This gave 2-chloro-8-trifluoroacetyl-7,8,9,10-tetrahydro-6H-azepino[4,5-g]quinoline (297 mg), a 30 mg (91 μmol) sample of which was dissolved in 1:1 THF/water (1 mL), treated with potassium carbonate (10 mg, 72 μmol) and stirred at ambient temperature for 48 h. The volatiles were removed by rotary evaporation, and the residue was purified by preparative HPLC, using mixtures of acetonitrile and 0.05% aqueous trifluoroacetic acid as mobile phase. Concentration of selected fractions gave 2-chloro-7,8,9,10-tetrahydro-6H-azepino[4,5-g]quinoline trifluoroacetate (8.3 mg, 39% yield) as white solid. ¹H NMR (CD₃OD, 300 MHz): δ8.25 (s, 1H), 7.80 (m, 2H), 7.45 (s, 1H), 3.45-3.25 (m, 8H). (MS m/z: 233 (M+H).
2-Methoxy-7,8,9,10-tetrahydro-6H-azepino[4,5-g]quinoline
A solution of 2-chloro-8-trifluoroacetyl-7,8,9,10-tetrahydro-6H-azepino[4,5-g]quinoline (30 mg, 91 μmol) in methanol (1 mL) was treated with sodium methoxide (0.5 mL of 25% solution, ˜2 mmol). The reaction mixture was refluxed for 6 h and cooled to ambient temperature. The volatiles were removed, and the residue was partitioned between ethyl acetate (5 mL) and water (1 mL). The aqueous layer was extracted with ethyl acetate (2×3 mL), and the combined organic extracts were dried over sodium sulfate and concentrated. The residue was purified by preparative HPLC, using mixtures of acetonitrile and 0.05% aqueous trifluoroacetic acid as mobile phase, to give 2-methoxy-7,8,9,10-tetrahydro-6H-azepino[4,5-g]quinoline trifluoroacetate (4.9 mg, 23% yield) as a syrup. ¹H NMR (CD₃OD, 300 MHz): δ8.05 (d, 1H), 7.68 (s, 1H), 7.64 (s, 1H), 6.93 (d, 1H), 4.0 (s, 3H), 3.4-3.2 (m, 8H). MS (m/z): 229 (M+H).
2-Methyl-7,8,9,10-tetrahydro-6H-azepino[4,5-g]quinoline
A mixture of 2-chloro-8-trifluoroacetyl-7,8,9,10-tetrahydro-6H-azepino[4,5-g]quinoline (30 mg, 91 μmol), tetrakis(triphenylphosphine)palladium(0) (10 mg, 8.7 μmol), tetramethyltin (25 μL, 180 μmol) and potassium carbonate (100 mg, 0.72 mmol) in toluene (1 mL) was refluxed for 48 h and cooled to ambient temperature. The volatiles were removed by rotary evaporation, and the residue was purified by preparative HPLC, using mixtures of acetonitrile and 0.05% aqueous trifluoroacetic acid as mobile phase. Selected fractions were concentrated, and the residue was dissolved in methanol (1 mL) and treated with potassium carbonate (10 mg, 72 μmol). This mixture was stirred for 3 h. The solids were then removed by filtration, and the residue was purified by preparative HPLC, using mixtures of acetonitrile and 0.05% aqueous trifluoroacetic acid as mobile phase. Concentration of selected fractions gave (3.5 mg, 18% yield) of 2-methyl-7,8,9,10-tetrahydro-6H-azepino[4,5-g]quinoline trifluoroacetate. ¹H NMR (CD₃OD, 300 MHz): δ8.05 (d, 1H), 7.64 (s, 1H), 7.58 (s, 1H), 7.32 (d, 1H), 3.2-2.85 (m, 8H), 2.62 (s, 3H). MS (m/z): 213 (M+H).

### Example 5

Example 5 describes a second generation synthesis of 7,8,9,10-tetrahydro-6H-azepino[4,5-g]quinoxaline and the synthesis of certain salts thereof.

7-Nitro-2,3,4,5-tetrahydro-1H-3-benzazepinium hydrogensulfate

Using an addition funnel, 2,3,4,5-tetrahydro-1H-3-benzazepine (112 g, 0.762 mol) was added drop-wise, over a 20 min period, to stirred and cooled (0-5° C.) trifluoroacetic acid (0.400 L, 5.42 mol) in a 2 L reactor. To the resulting solution, 98% sulfuric acid (0.150 L, 2.78 mol) was added over a 10 min period (using the addition funnel), while keeping the temperature below 10° C. Similarly, fuming nitric acid (0.050 L of >90%, ˜1.2 mol) was added drop-wise over a 40 min period (using the addition funnel), while keeping the temperature below 10° C. (Caution: The nitric acid addition was strongly exothermic). The resulting solution was stirred at 0° C. and sampled every 30 min for completion (LCMS evidence of disappearance of starting material). After a total of 60 min of stirring, ethyl acetate (0.5 L) was slowly added (through the addition funnel), producing a precipitate. Another 1.5 L of ethyl acetate was then added in one portion, and the mixture was stirred at 20-25° C. for 30 min. The solids were collected by suction filtration, washed with ethyl acetate (3×0.50 L), washed with hexanes (2×0.60 L), and air dried for 2 h. The resulting off-white solid weighed 190 g (86% yield). ¹H NMR (D₂O, 300 MHz): δ7.85-7.79 (2H, m), 7.24-7.20 (1H, m), 3.22-3.14 (4H, m), 3.13-3.05 (4H, m). MS m/z: 193 (M +H). MP: 237-240° C. with decomposition.

7-Nitro-3-trifluoroacetyl-2,3,4,5-tetrahydro-1H-3-benzazepine

Chloroform (1.10 L) and 7-nitro-2,3,4,5-tetrahydro-1H-3-benzazepinium hydrogensulfate salt (232 g, 0.801 mol) were placed in a 3 L reactor. This mixture was stirred and cooled (5-10° C.) as aqueous sodium hydroxide (1.00 L of 10 wt %, 100 g, 2.50 mol) was added via an addition funnel. This biphasic mixture was stirred vigorously for 30 min and allowed to stand. The bottom layer was collected and dried over anhydrous sodium sulfate (130 g) for 1-2 h. The drying agent was removed by suction filtration, and the filter cake was rinsed with chloroform (0.10 L). The combined filtrates were concentrated under reduced pressure at 40-50° C., leaving a viscous oil (148 g). This was dissolved in tetrahydrofuran (0.50 L), transferred to a 2 L reactor, stirred and cooled to 10-15° C. Triethylamine (0.280 L, 2.02 mol) was added to the solution, followed by drop-wise addition (via addition funnel) of trifluoroacetic anhydride (0.14 L, 0.99 mol) over a 20 min period, keeping the reaction temperature between 15-30° C. Stirring was continued until LCMS analysis indicated that the reaction was complete (disappearance of starting material). Hydrochloric acid (0.70 L of 1 N, 0.70 mol) was then added as a thin stream via dropping addition funnel over 10 min period. This addition produced a temperature rise from 25° C. to 37° C. The product initially oiled out. Seed crystals (200 mg) were added, and stirring was continued for 30 min, during which time the oil became a free flowing solid. The suspension was cooled to 5° C., stirred at that temperature for 1 h and suction filtered to collect the solids. The filter cake was washed with methanol (2×0.30 L), washed with hexanes (2×0.50 L) and air-dried for 3 h. The resulting off-white solid weighed 227 g (of 97.7% purity- (96% yield). ¹H NMR (CDCl₃, 300 MHz): 8 8.06-8.02 (2H, m), 7.24-7.20 (1H, m), 3.84-3.80 (2H, m), 3.77-3.74 (2H, m), 3.13-3.09 (4H, m). MP: 129-132° C.

7-Amino-3-trifluoroacetyl-2,3,4,5-tetrahydro-1H-3-benzazepine

7-Nitro-3-trifluoroacetyl-2,3,4,5-tetrahydro-1H-3-benzazepine (103 g of 97.7%, 0.349 mol), methanol (1.0 L) and zinc (110 g, 1.68 mol) were placed in a 2 L reactor. This suspesion was stirred and cooled to 0° C. Saturated aqueous ammonium chloride (112 g in 0.30 L de-ionized water, 2.11 mol) was added (via addition funnel) as a thin stream over a 10 min period. The temperature climbed steadily from 0° C. to 45° C. during the addition. The suspension was then heated at 62-65° C. for ˜1 h, until LCMS analysis indicated that starting material was gone. The reaction mixture was cooled to ambient temperature and suction filtered through a 1 μm pad. The wet cake was washed with methanol (2×0.15 L), and the combined filtrates were concentrated under reduced pressure at 50-55° C. The semisolid concentrate was dissolved in chloroform (0.50 L) and transferred back to the reactor, where it was stirred as a 1:1 mixture of sodium bicarbonate and deionized water (0.20 L). After vigorous stirring for 5 min, the phases were allowed to separate, and the bottom (chloroform) layer was drawn off. The aqueous layer was returned to the reactor, combined with chloroform (0.30 L) and stirred vigorously. Again the bottom layer was drawn off, and the combined chloroform layers were dried over anhydrous sodium sulfate (100 g) for 2 h. After suction filtration (to remove the drying agent) and concentration of the filtrate under reduced pressure at 55-60° C., a light yellow solid (87.0 g, 97% yield) remained. ¹H NMR (CDCl₃, 300 MHz): 5 6.93-6.83 (1H, m), 6.50-6.46 (2H, m), 3.75-3.70 (2H, m), 3.67-3.62 (4H, m), 2.94-2.83 (4H, m). MP: 116-118° C.

N-(3-(Trifluoroacetyl)-2,3,4,5-tetrahydro-1H-3-benzazepin-7-yl)acetamide
A mixture of 7-amino-3-trifluoroacetyl-2,3,4,5-tetrahydro-1H-3-benzazepine (167 g, 0.647 mol), 2-methyltetrahydrofuran (2.0 L), and triethylamine (115 mL, 0.831 mol), in a 3 L reactor, was warmed to 50-55° C. to generate a solution. The solution was then cooled the solution to 0° C., and acetyl chloride (50 mL, 0.70 mol) was added drop-wise via an addition funnel over a 15 min period. The resulting suspension was stirred for 30 min, at which time the reaction was complete by LCMS analysis. Hydrochloric acid (0.50 L of 1 N, 0.50 mol) was then added, and the biphasic mixture was stirred for 10 min. The phases were allowed to separate, and the top (organic) layer was removed. The aqueous layer was returned to the reactor and stirred with ethyl acetate (0.70 L) for 10 min. Again the phases were allowed to separate, and the aqueous (bottom layer was removed. The organic layers were then combined and stirred with hydrochloric aid (0.20 L of 1 N) for 10 min. The top (organic) layer was removed and dried over anhydrous sodium sulfate (100 g) for 60 min. Suction filtration (to remove the drying agent) and concentration of the filtrate under reduced pressure at 50-55° C. produced a solid. The solid was triturated with hexanes (1.00 L) for 30 min and collected by suction filtration. After washing with hexanes (2×0.30 L) and air drying for 2-3 h, the solid was ground into a fine powder with a mortar and pestle.

Heating at 60-65° C. under reduced pressure for 1 h left off-white solids weighing 186 g of 94% purity (90% yield). ¹H NMR (CDCl₃, 300 MHz): δ7.94-7.85 (1H bs), 7.45-7.37 (1H, dd), 7.30-7.21 (1H, dd), 7.07-7.05 (1H, m), 3.75-3.71 (2H, m), 3.68-3.65 (2H, m), 2.95-2.88 (4H, m), 2.16 (3H, s). MS m/z: 301 (M +H). MP: 174-176° C.

N-(8-Nitro-3-(trifluoroacetyl)-2,3,4,5-tetrahydro-1H-3-benzazepin-7-yl)acetamide
To a 1 L reactor containing concentrated sulfuric acid (0.80 L, 15.0 mol), stirred and cooled to 0° C., was added N-(3-(trifluoroacetyl)-2,3,4,5-tetrahydro-1H-3-benzazepin-7-yl)acetamide (112 g of 94%, 0.35 mol) as a solid over a period of 5 min. Stirring was continued at 0-5° C. until solution was obtained (˜40 min). Fuming nitric acid (19 mL of >90%, 0.44 mol) drop-wise from an addition funnel over a 45 min period, keeping the temperature below 2° C. The resulting yellow/orange solution was stirred for 60 min at 0-5° C., at which time LCMS analysis indicated that the reaction was complete. The reaction mixture was slowly poured into ice (1.3 kg) in a 4 L flask. This mixture was stirred for 20 min, and then dichloromethane (0.60 L) was added. After stirring vigorously for 15 min, the phases were allowed to separate. The dichloromethane layer was collected, and the aqueous layer was returned to the flask and combined with a second portion of dichloromethane (0.60 L).

After 10 min of thorough mixing, the phases were allowed to separate, and the dichloromethane layer was drawn off. The combined dichloromethane layers were returned to the flask and stirred with saturated aqueous sodium bicarbonate (0.60 L) for 10 min. The dichloromethane layer was drawn off and dried over anhydrous sodium sulfate (100 g) for 60 min. The drying agent was removed by suction filtration, and the filtrate was concentrated under reduced pressure at 50-55° C., leaving a viscous oil. The oil was dissolved in methanol (0.20 L) and concentrate under reduced pressure at 50-55° C., producing a solid. The solid was triturated with hexanes (0.50 L) for 30 min at 40-50° C. and collected by suction filtration. Washing with hexanes (2×0.10 L), followed by air drying for 2-3 h, left 113 g of yellow solid, which is a mixture of the desired 8-nitro and the byproduct 6-nitro regioisomers in a ratio of 3:1 by NMR analysis (71% yield of desired isomer). 8-Nitro regiosiomer ¹H NMR (CDCl₃, 300 MHz): δ10.30 (1H, bs), 8.65-8.63 (1H d), 8.01-7.98 (1H, d), 3.81-3.78 (2H, m), 3.75-3.71 (2H, m), 3.08-3.02 (4H, m), 2.95 (3H, s). 6-Nitro regioisomer ¹H NMR (CDCl₃, 300 MHz): δ8.05-8.00 (1H bs), 7.96-7.90 (1H m), 7.32-7.37 (1H, m), 3.81-3.78 (2H, m), 3.75-3.71 (2H, m), 3.08-3.02 (2H, m), 2.90-2.88 (2H, m) 2.20 (3H, s).

tert-Butyl 7-amino-8-nitro-1,2,4,5-tetrahydro-3H-3-benzazepine-3-carboxylate
A mixture of N-(8-nitro-3-(trifluoroacetyl)-2,3,4,5-tetrahydro-1H-3-benzazepin-7-yl)acetamide (310 g of 75%, 0.674 mol) and methanol (0.80 L), in a 3 L reactor, was stirred and heated to 55-60° C. To the warm solution, solid potassium carbonate (251 g, 1.82 mol) was added in portions over a 5 min period (the reaction temperature rose ˜5° C. during the addition). The reaction mixture was kept at 55-60° C. for 1 h (at which time LCMS analysis indicated that the reaction was complete) and then cooled to ambient temperature. Diatomaceous earth (100 g) was added, and the mixture was stirred for 10 min and suction filtered. The filter cake was washed with methanol (0.20 L), and the filtrate was returned to the 3 L reactor and cooled to 10° C. With stirring, solid di-tert-butyl dicarbonate (179 g, 0.820 mol) was added in portions (off-gassing and mild exotherm occur). The reaction mixture was stirred at 15-20° C. for 5 h, during which time a precipitate formed. LCMS analysis indicated that the reaction was complete, so the reactor was cooled to 0-5° C. for 2 h and suction filtered. The filter cake was washed with methanol (2×0.15 L), washed with hexanes (2×0.20 L) and air dried for 2 h, leaving 131 g of yellow solid. NMR analysis indicated that this material is the desired 8-nitro regioisomer only. ¹H NMR (CDCl₃, 300 MHz): δ7.86 (1H s), 6.59 (1H, s), 6.06 (2H, bs), 3.54-3.51 (4H, m), 2.82-2.81 (4H, m), 1.49 (9H, s). MS m/z: 208 (M+1-t-butoxycarbonyl). MP: 160-162° C.

A second crop was obtained as follows: The filtrate was concentrated under reduced pressure at 55-60° C., and the resulting semi-solid was partitioned between 5% aqueous sodium hydroxide (1.0 L) and ethyl acetate (0.70 L) (thorough mixing for 5 min, followed by separation of the phases). The organic phase was dried over anhydrous sodium sulfate (100 g) for 30 min, filtered and concentrated under reduced pressure at 55-60° C. to obtain a viscous oil (130 g). This was dissolved in methanol (0.20 L) to the viscous oil and concentrate again to remove the ethyl acetate (and most of the methanol). The viscous solution was cooled to ambient temperature and diluted with methanol (0.080 L). The walls of the flask were scraped to induce crystallization, and then the mixture was stirred for 5 h. The solids were collected by suction filtration, washed with methanol (2×20 mL), washed with hexanes (2×50 mL) and air dried for 2 h, yielding another 5 g of yellow solid (8-nitro regioisomer). Total yield=136 g (66%).

tert-Butyl 7,8-diamino-1,2,4,5-tetrahydro-3H-3-benzazepine-3-carboxylate

A stirred mixture of tert-butyl 7-amino-8-nitro-1,2,4,5-tetrahydro-3H-3-benzazepine-3-carboxylate (158 g, 0.515 mol), zinc (158 g, 2.42 mol) and methanol (1.58 L) in a 3 L reactor was cooled to 10° C. To this was added, as a thin stream from an addition funnel, saturated aqueous ammonium chloride (153 g, 2.86 mol in 0.45 L of de-ionized water). The addition took 10 min, and the temperature of the reaction climbed from 9° C. to 45° C. over the course of the addition. The suspension was then warmed to 60-65° C. and held at that temperature for 1 h, at which point LCMS analysis indicated that the reaction was complete (Note: the color of the suspension changes from deep orange to pale yellow during heating). The reaction mixture was cooled ambient temperature and suction filtered. The filter cake was washed with methanol (2×0.30 L), and the filtrate was concentrated under reduced pressure at 55-60° C. The semisolid concentrate was diluted with dichloromethane (0.80 L) and mixed, in the 3 L reactor, with 5% aqueous sodium hydroxide (0.25 L) for 5 min. After allowing the phases to separate, the bottom (organic) layer was drawn off and dried over anhydrous sodium sulfate (100 g) for 1 h. The drying agent was removed by suction filtration, and the filtrate was concentrated under reduced pressure at 55-60° C., leaving a tan solid (138 g of 98% purity; 94% yield). ¹H NMR (CDCl₃, 300 MHz): δ6.47 (2H s), 3.49 (4H, m), 3.30 (4H,bs), 2.72 (4H, m), 1.48 (9H, s). MS m/z: 178 (M+1-t-butoxycarbonyl). MP: 147-149° C.

tert-Butyl 6,7,9,10-tetrahydro-8H-azepino[4,5-g]quinoxaline-8-carboxylate

A mixture of tert-butyl 7,8-diamino-1,2,4,5-tetrahydro-3H-3-benzazepine-3-carboxylate (254 g of 98%, 0.90 mol) and isopropyl alcohol (1.80 L) was stirred in a 3 L reactor and heated to 65-70° C. until a red solution formed (required ˜30 min). The solution was cooled to 25-28° C., and water (0.45 L) and p-dioxane-2,3-diol (110 g, 0.915 mol) were added. The temperature rose from 28° C. to 32° C. over a 15 min period, stayed at 32° C. for 15 min, then slowly dropped to ambient temperature. After stirring at ambient temperature for 1 h, LCMS analysis indicated that the reaction was complete. The reaction mixture was concentrated under reduced pressure at 50-55° C. The resulting viscous mass (˜0.60 L) was stirred with de-ionized water (0.55 L) for 30 min. The resulting suspension was suction filtered, and the filter cake was washed with de-ionized water (3×0.55 L). The collected solids were returned to the reactor and mixed with dichloromethane (0.60 L) for 5 min. The phases were allowed to separate, and the bottom (organic) layer was dried over anhydrous sodium sulfate (100 g) for 1 h. The drying agent was removed by suction filtration and washed with dichloromethane (0.20 L). The combined filtrates were concentrated under reduced pressure at 50-60° C., producing a light tan solid (250 g of 99% purity; 92% yield). ¹H NMR (CDCl₃, 300 MHz): 8 8.77 (2H s), 7.84 (2H, s), 3.67-3.65 (4H, m), 3.15-3.13 (4H, m), 1.50 (9H, s). MP: 107-109° C.

7,8,9,10-Tetrahydro-6H-azepino[4,5-g]quinoxaline

tert-Butyl 6,7,9,10-tetrahydro-8H-azepino[4,5-g]quinoxaline-8-carboxylate (250 g of 99%, 0.826 mol) was added in portions, with stirring over a 5 min period, to concentrated hydrochloric acid (0.60 L, 7.31 mol) in a water bath cooled 3 L reactor. The solids dissolved upon contact with the hydrochloric acid solution, with off-gassing noted. This solution was stirred at ambient temperature for 30 min, at which point LCMS analysis indicated that the reaction was complete. The reaction was then cooled to below 5° C., and aqueous sodium hydroxide (550 g of 50wt %, 6.9 mol) was added, as a thin stream from an addition funnel, over a 20 min period. The resulting solution had a pH>12. Chloroform (1.00 L) was added, and the biphasic mixture was stirred vigorously for 10 min and suction filtered through a pad of diatomaceous earth (50 g). A small amount of residue resides on the diatomaceous earth (chloroform and water insoluble). The dark red chloroform layer was collected, and the aqueous layer was stirred with a second portion of chloroform (1.00 L) for 10 min. Again the biphasic mixture was suction filtered through the diatomaceous earth pad and the chloroform layer was collected. The combined chloroform layers were dried over anhydrous sodium sulfate (100 g) for 60 min, suction filtered to remove the drying agent, and concentrated under reduced pressure at 50-60° C. The resulting solid was triturated with heptane (0.30 L) at 60-65° C. for 30 min, before removing the heptanes under reduced pressure at 40-45° C. During the drying process, the solids were ground into a powder. The resulting light beige solid weighed 163 g and was a hydrated form of the free base as evidenced by ¹H NMR (between 0.5 and 1 equivalents of water). ¹H NMR (CDCl₃, 300 MHz): δ8.76 (2H s), 7.80 (2H, s), 3.17-3.14 (4H, m), 3.07-3.05 (4H, m), 1.98 (>1H, bs). MS m/z: 200 (M +1). MP: 114-116° C.

7,8,9,10-Tetrahydro-6H-azepino[4,5-g]quinoxalin-8-ium succinate

A mixture of succinic acid (141 g, 1.20 mol) and absolute ethanol (2.30 L) was heated to 73-75° C. in a 5 L flask. To this solution was added (as a thin stream from an addition funnel over a 45 min period) a warm (45-50° C.) solution of 7,8,9,10-tetrahydro-6H-azepino[4,5-g]quinoxaline (239 g, 1.20 mol) in absolute ethanol (0.70 L). The salt began precipitating early during the addition. The addition funnel was washed with absolute ethanol (0.40 L) which was then added to the reaction. De-ionized water (30 mL) was added, and the mixture was stirred at 73-75° C. for 30 min, cooled to ambient temperature gradually and stirred for there for 1 h. The solid was collected by suction filtration, washed with ethanol (3×0.50 L), washed with hexanes (3×0.70 L), and air dried for 1.5 h. Vacuum oven drying, at 75° C. overnight at 20 inches of Hg with a nitrogen sweep, left a light tan powder (362 g; 95.3% yield). ¹H-NMR (DMSO-d₆, 300 MHz): δ8.87 (2H s), 7.89 (2H, s), 3.24-3.21 (4H, m), 3.11-3.08 (4H, m), 2.33 (4H, s). ¹H-NMR (D₂O, 300 MHz): δ8.52 (2H s), 7.39 (2H, s), 3.24-3.22 (4H, m), 3.13-3.10 (4H, m), 2.32 (4H, s). ¹H-NMR (DMSO and D₂O) show no regioisomer or other impurities present. HPLC purity is 99.8%. MP: 223-224° C.

7,8,9,10-Tetrahydro-6H-azepino[4,5-g]quinoxalin-8-ium L-tartrate

A stirred suspension of L-tartaric Acid (141 mg, 0.940 mmol) in absolute ethanol (2.5 mL) was heated to near boiling until complete dissolution of the acid was attained. To the hot acid solution was added a solution of 7,8,9,10-tetrahydro-6H-azepino[4,5-g]quinoxaline (187 mg, 0.940 mmol) in ethanol (2.5 mL) drop-wise over a 5 min period, followed by addition of a 0.5 mL ethanol rinse of the amine vessel. The product precipitated immediately. Upon completion of amine addition, the reaction mixture was brought to reflux for 1 min, and then cooled slowly to ambient temperature (22° C.) over 3 h. The resulting product was collected by vacuum filtration, washed generously with ethanol, and then dried under nitrogen cone for 1 h to give 265 mg of off-white powder (MP=221-222° C., with decomposition). ¹H-NMR was consistent with 1:1 stochiometry. ¹H-NMR (DMSO-d₆, 400 MHz): δ8.91 (2H s), 7.94 (2H, s), 3.95 (2H, s), 3.35-3.30 (4H, m), 3.28-3.23 (4H, m).

7,8,9,10-Tetrahydro-6H-azepino[4,5-g]quinoxalin-8-ium hydroxybenzoate

A stirred solution of 4-hydroxybenzoic acid (121 mg, 0.873 mmol) in absolute ethanol (2 mL) was heated to near boiling. A solution of 7,8,9,10-tetrahydro-6H-azepino[4,5-g]quinoxaline (174 mg, 0.873 mmol) in absolute ethanol (1 mL) was then added drop-wise over 5 min while maintaining the solution temperature near boiling. The solution was brought to ambient temperature and stirred for 1 h. Hexanes (1 mL) were then added drop-wise until a permanent turbidity was attained. Stirring was continued, resulting in the precipitation of fine solids. The stirred mixture was warmed to ˜55° C. and diluted further with hexane (1.5 mL). The mixture was then cooled to ambient temperature (22° C.) and allowed to stand overnight (16 h) without stirring. The resulting solids were collected by vacuum filtration, washed with hexanes, and dried under nitrogen cone for 1 h to give 262 mg of a very faintly yellow powder with (MP=212-213° C.). ¹H-NMR was consistent with 1:1 stochiometry. ¹H-NMR (DMSO-d₆, 400 MHz): δ8.85 (2H s), 7.84 (2H, s), 7.77 (2H, d), 6.80 (2H, d), 3.17-3.12 (4H, m), 2.96-2.91 (4H, m).

7,8,9,10-Tetrahydro-6H-azepino[4,5-g]quinoxalin-8-ium sesquihydrochloride

Potassium carbonate (186 mg, 1.35 mmol) was added to a solution of 8-trifluoroacetyl-7,8,9,10-tetrahydro-6H-azepino[4,5-g]quinoxaline (199 mg, 0.670 mmol) in anhydrous methanol (5 mL) and stirred at ambient temperature for 16 h. The solids were removed by vacuum filtration, and the filtrate was concentrated and purified by preparative HPLC, using mixtures of acetonitrile and 0.05% aqueous trifluoroacetic acid as mobile phase. The resulting trifluoroacetate salt was converted into free base by partitioning between chloroform (10 mL) and 20% aqueous potassium carbonate (5 mL). The aqueous layer extracted with chloroform (2×10 mL), and the combined organic extracts were dried over anhydrous sodium sulfate, filtered and concentrated. The residue was dissolved in methanol (3 mL) and combined with 4M hydrochloric acid in dioxane (3 mL). This mixture was concentrated, re-dissolved in methanol and concentrated again (several cycles) resulting in the azeotropic removal of excess hydrochloric acid. The resulting 7,8,9,10-tetrahydro-6H-azepino[4,5-g]quinoxaline sesquihydrochloride (70 mg, 41%) was a brown solid. ¹H-NMR (D₂O, 300 MHz): δ8.52 (2H s), 7.27 (2H, s), 3.20-3.14 (4H, m), 3.07-3.00 (4H, m). Ion chromatography indicated the presence of 1.5 equivalents of hydrochloric acid.

### VIII. Biological Assays

### Example 6: Radioliaand Bindina at CNS nAChRs

α4β2 nAChR Subtype

Preparation of membranes from rat cortex: Rats (female, Sprague-Dawley), weighing 150-250 g, were maintained on a 12 h light/dark cycle and were allowed free access to water and food supplied by PMI Nutrition International, Inc. Animals were anesthetized with 70% CO₂, and then decapitated. Brains were removed and placed on an ice-cold platform. The cerebral cortex was removed and placed in 20 volumes (weight:volume) of ice-cold preparative buffer (137 mM NaCl, 10.7 mM KCl, 5.8 mM KH₂PO₄, 8 mM Na₂HPO₄, 20 mM HEPES (free acid), 5 mM iodoacetamide, 1.6 mM EDTA, pH 7.4); PMSF, dissolved in methanol to a final concentration of 100 μM, was added and the suspension was homogenized by Polytron. The homogenate was centrifuged at 18,000×g for 20 min at 4° C. and the resulting pellet was re-suspended in 20 volumes of ice-cold water. After 60 min incubation on ice, a new pellet was collected by centrifugation at 18,000×g for 20 min at 4° C. The final pellet was re-suspended in 10 volumes of buffer and stored at ˜20° C.

Preparation of membranes from SH-EP1/human α4β2 clonal cells:

Cell pellets from 40 150 mm culture dishes were pooled, and homogenized by Polytron (Kinematica GmbH, Switzerland) in 20 milliliters of ice-cold preparative buffer. The homogenate was centrifuged at 48,000 g for 20 minutes at 4° C. The resulting pellet was re-suspended in 20 mL of ice-cold preparative buffer and stored at ˜20° C.

On the day of the assay, the frozen membranes were thawed and spun at 48,000×g for 20 min. The supernatant was decanted and discarded. The pellet was resuspended in Dulbecco's phosphate buffered saline (PBS, Life Technologies) pH 7.4 and homogenized with the Polytron for 6 seconds. Protein concentrations were determined using a Pierce BCA Protein Assay Kit, with bovine serum albumin as the standard (Pierce Chemical Company, Rockford, Ill.).

Assay: Membrane preparations (approximately 50 μg for human and 200-300 μg protein for rat α4β2) were incubated in PBS (50 μL and 100 μL respectively) in the presence of competitor compound (0.01 nM to 100 μM) and 5 nM [³H]nicotine for 2-3 hours on ice. Incubation was terminated by rapid filtration on a multi-manifold tissue harvester (Brandel, Gaithersburg, Md.) using GF/B filters presoaked in 0.33% polyethyleneimine (w/v) to reduce non-specific binding. Tissue was rinsed 3 times in PBS, pH 7.4. Scintillation fluid was added to filters containing the washed tissue and allowed to equilibrate. Filters were then counted to determine radioactivity bound to the membranes by liquid scintillation counting (2200CA Tri-Carb LSC, Packard Instruments, 50% efficiency or Wallac Trilux 1450 MicroBeta, 40% efficiency, Perkin Elmer).

Data were expressed as disintegrations per minute (DPMs). Within each assay, each point had 2-3 replicates. The replicates for each point were averaged and plotted against the log of the drug concentration. IC₅₀, which is the concentration of the compound that produces 50% inhibition of binding, was determined by least squares non-linear regression. Ki values were calculated using the Cheng-Prussof equation (1973):

Ki=IC₅₀/(1+N/Kd)

where N is the concentration of [³H]nicotine and Kd is the affinity of nicotine (3 nM, determined in a separate experiment).

α7 nAChR Subtype

Preparation of membranes from rat hippocampus: Rats (female, Sprague-Dawley), weighing 150-250 g, were maintained on a 12 h light/dark cycle and were allowed free access to water and food supplied by PMI Nutrition International, Inc. Animals were anesthetized with 70% CO₂, then decapitated. Brains were removed and placed on an ice-cold platform. The hippocampus was removed and placed in 10 volumes (weight:volume) of ice-cold preparative buffer (137 mM NaCl, 10.7 mM KCl, 5.8 mM KH₂PO₄, 8 mM Na₂HPO₄, 20 mM HEPES (free acid), 5 mM iodoacetamide, 1.6 mM EDTA, pH 7.4); PMSF, dissolved in methanol to a final concentration of 100 μM, was added and the tissue suspension was homogenized by Polytron. The homogenate was centrifuged at 18,000×g for 20 min at 4° C. and the resulting pellet was re-suspended in 10 volumes of ice-cold water. After 60 min incubation on ice, a new pellet was collected by centrifugation at 18,000×g for 20 min at 4° C. The final pellet was re-suspended in 10 volumes of buffer and stored at ˜20° C.

On the day of the assay, tissue was thawed, centrifuged at 18,000×g for 20 min, and then re-suspended in ice-cold PBS (Dulbecco's Phosphate Buffered Saline, 138 mM NaCl, 2.67 mM KCl, 1.47 mM KH₂PO₄, 8.1 mM Na₂HPO₄, 0.9 mM CaCl₂, 0.5 mM MgCl₂, Invitrogen/Gibco, pH 7.4) to a final concentration of approximately 2 mg protein/mL. Protein was determined by the method of Lowry et al., J. Biol. Chem. 193: 265 (1951), using bovine serum albumin as the standard.

Assay: The binding of [³H]MLA was measured using a modification of the methods of Davies et al., Neuropharmacol. 38: 679 (1999). [³H]MLA (Specific Activity=25-35 Ci/mmol) was obtained from Tocris. The binding of [³H]MLA was determined using a 2 h incubation at 21° C. Incubations were conducted in 48-well micro-titre plates and contained about 200 pg of protein per well in a final incubation volume of 300 μL. The incubation buffer was

PBS and the final concentration of [³H]MLA was 5 nM. The binding reaction was terminated by filtration of the protein containing bound ligand onto glass fiber filters (GF/B, Brandel) using a Brandel Tissue Harvester at room temperature. Filters were soaked in de-ionized water containing 0.33% polyethyleneimine to reduce non-specific binding. Each filter was washed with PBS (3×1 mL) at room temperature. Non-specific binding was determined by inclusion of 50 μM non-radioactive MLA in selected wells.

The inhibition of [³H]MLA binding by test compounds was determined by including seven different concentrations of the test compound in selected wells. Each concentration was replicated in triplicate. IC₅₀ values were estimated as the concentration of compound that inhibited 50 percent of specific [³H]MLA binding. Inhibition constants (Ki values), reported in nM, were calculated from the IC₅₀ values using the method of Cheng et al., Biochem. Pharmacol. 22: 3099-3108 (1973).

### Example 7: Interaction at the Human Muscle nAChR Subtype

Activation of muscle-type nAChRs was established on the human clonal line TE671/RD, which is derived from an embryonal rhabdomyosarcoma (Stratton et al., Carcinogen 10: 899 (1989)). These cells express receptors that have pharmacological (Lukas, J. Pharmacol. Exp. Ther. 251: 175 (1989)), electrophysiological (Oswald et al., Neurosci. Lett. 96: 207 (1989)), and molecular biological profiles (Luther et al., J. Neurosci. 9: 1082 (1989)) similar to the muscle-type nAChR.

TE671/RD cells were maintained in proliferative growth phase according to routine protocols (Bencherif et al., Mol. Cell. Neurosci. 2: 52 (1991) and Bencherif et al., J. Pharmacol. Exp. Ther. 257: 946 (1991)). Cells were cultured in Dulbecco's modified Eagle's medium (Gibco/BRL) with 10% horse serum (Gibco/BRL), 5% fetal bovine serum (HyClone, Logan Utah), 1 mM sodium pyruvate, 4 mM L-Glutamine, and 50,000 units penicillin-streptomycin (Irvine Scientific). When cells were 80% confluent, they were plated to 12 well polystyrene plates (Costar). Experiments were conducted when the cells reached 100% confluency.

Nicotinic acetylcholine receptor (nAChR) function was assayed using ⁸⁶Rb⁺efflux according to the method described by Lukas et al., Anal. Biochem. 175: 212 (1988). On the day of the experiment, growth media was gently removed from the well and growth media containing ⁸⁶Rubidium chloride (10⁶ μCi/mL) was added to each well. Cells were incubated at 37° C. for a minimum of 3 h. After the loading period, excess ⁸⁶Rb⁺was removed and the cells were washed twice with label-free Dulbecco's phosphate buffered saline (138 mM NaCl, 2.67 mM KCl, 1.47 mM KH₂PO₄, 8.1 mM Na₂HPO₄, 0.9 mM CaCl₂, 0.5 mM MgCl₂, Invitrogen/Gibco, pH. 7.4), taking care not to disturb the cells. Next, cells were exposed to either 100 μM of test compound, 100 μM of L-nicotine (Acros Organics) or buffer alone for 4 min. Following the exposure period, the supernatant containing the released ⁸⁶Rb⁺was removed and transferred to scintillation vials. Scintillation fluid was added and released radioactivity was measured by liquid scintillation counting.

Within each assay, each point had 2 replicates, which were averaged. The amount of ⁸⁶Rb⁺release was compared to both a positive control (100 μM L-nicotine) and a negative control (buffer alone) to determine the percent release relative to that of L-nicotine.

When appropriate, dose-response curves of test compound were determined. The maximal activation for individual compounds (Emax) was determined as a percentage of the maximal activation induced by L-nicotine. The compound concentration resulting in half maximal activation (EC₅₀) of specific ion flux was also determined.

### Example 8: Interaction at the Human Ganglionic nAChR Subtype

The cell line SH-SY5Y is a continuous line derived by sequential subcloning of the parental cell line, SK-N-SH, which was originally obtained from a human peripheral neuroblastoma. SH-SY5Y cells express a ganglion-like nAChR (Lukas et al., Mol. Cell. Neurosci. 4: 1 (1993)).

Human SH-SY5Y cells were maintained in proliferative growth phase according to routine protocols (Bencherif et al., Mol. Cell. Neurosci. 2: 52 (1991) and Bencherif et al., J. Pharmacol. Exp. Ther. 257: 946 (1991)). Cells were cultured in Dulbecco's modified Eagle's medium (Gibco/BRL) with 10% horse serum (Gibco/BRL), 5% fetal bovine serum (HyClone, Logan Utah), 1 mM sodium pyruvate, 4 mM L-Glutamine, and 50,000 units penicillin-streptomycin (Irvine Scientific). When cells were 80% confluent, they were plated to 12 well polystyrene plates (Costar). Experiments were conducted when the cells reached 100% confluency.

Nicotinic acetylcholine receptor (nAChR) function was assayed using ⁸⁶Rb⁺efflux according to a method described by Lukas et al., Anal. Biochem. 175: 212 (1988). On the day of the experiment, growth media was gently removed from the well and growth media containing ⁸⁶Rubidium chloride (10⁶ μCi/mL) was added to each well. Cells were incubated at 37° C. for a minimum of 3 h. After the loading period, excess ⁸⁶Rb⁺was removed and the cells were washed twice with label-free Dulbecco's phosphate buffered saline (138 mM NaCl, 2.67 mM KCl, 1.47 mM KH₂PO₄, 8.1 mM Na₂HPO₄, 0.9 mM CaCl₂, 0.5 mM MgCl₂, Invitrogen/Gibco, pH 7.4), taking care not to disturb the cells. Next, cells were exposed to either 100 μM of test compound, 100 μM of nicotine, or buffer alone for 4 min. Following the exposure period, the supernatant containing the released ⁸⁶Rb⁺was removed and transferred to scintillation vials. Scintillation fluid was added and released radioactivity was measured by liquid scintillation counting.

Within each assay, each point had 2 replicates, which were averaged. The amount of ⁸⁶Rb⁺release was compared to both a positive control (100 μM nicotine) and a negative control (buffer alone) to determine the percent release relative to that of L-nicotine.

When appropriate, dose-response curves of test compound were determined. The maximal activation for individual compounds (Emax) was determined as a percentage of the maximal activation induced by L-nicotine. The compound concentration resulting in half maximal activation (EC₅₀) of specific ion flux was also defined.

### Tabular Receptor Binding Data

TABLE 1

| | | Human α4β2 | Rat α4β2 |
|---|---|---|---|
| Compound | Structure | Ki (nM) | Ki (nM) |
| A | 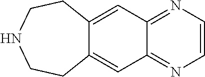 | 10 | 17 |
| B | 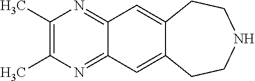 | 98 | 590 |
| C | 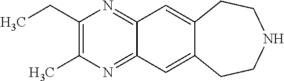 | 310 | Failed HTS |
| D | 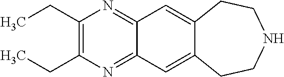 | 460 | Failed HTS |
| E | 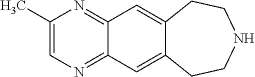 | 37 | 63 |
| F | 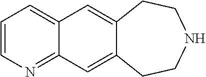 | 0.93 | 3 |
| G | 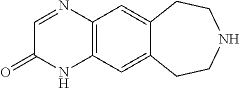 | 63 | 120 |
| H | 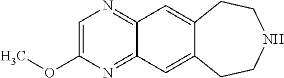 | 25 | 77 |
| I | 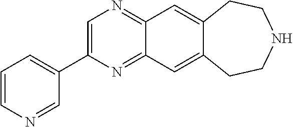 | 7400 | Failed HTS |
| J | 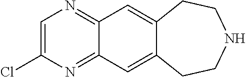 | 45 | 12 |
| K | 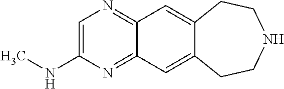 | Failed HTS | Failed HTS |
| L | 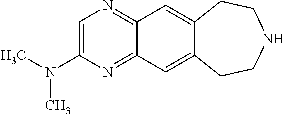 | 740 | Failed HTS |
| M | 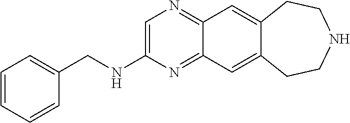 | 530 | Failed HTS |
| N | 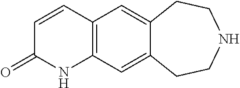 | Failed HTS | Failed HTS |
| O | 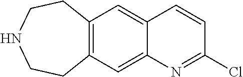 | 0.48 | 3.1 |
| P | 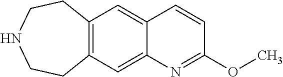 | 1200 | Failed HTS |
| Q | 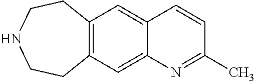 | 11 | 45 |
| R | 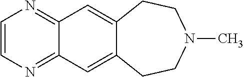 | 210 | Failed HTS |

### Summary of In Vitro Biological Data

Compounds recited in Table 1, representative of the present invention, generally exhibit inhibition constants (Ki values) at the rat and human α4β2 subtypes in the nanomolar to low micromolar range, indicating high affinity for the α4β2 subtype. In some cases, the compounds failed to bind sufficiently in high through-put screening (HTS) for the α4β2 subtype to warrant Ki determination. These are labeled “Failed HTS” in Table 1. Ki values at the α7 subtype were not determined, as the compounds of the present inventions consistently failed HTS at the α7 subtype. Likewise, preliminary results indicate that the compounds of the present invention do not interact significantly with human ganglionic and muscle nAChR subtypes, indicating that they will exhibit minimal unwanted nicotinic side effects. Thus, as an example, 7,8,9,10-tetrahydro-6H-azepino[4,5-g]quinoxaline exhibits an EC₅₀ of 4.4 micromolar and an Eₘₐₓ of 21% at the human ganglionic subtype and an EC₅₀ of 75 micromolar and an Eₘₐₓ of 23% at the human muscle subtype.

### Example 9: In vivo pharmacology: Novel Object Recognition

Episodic memory is a cognitive domain known to be impaired in Alzheimer's disease; the Novel Object Recognition task is a commonly used and quick, clean model used to assess potential cognitive benefit derived from the test compounds. Compound A (7,8,9,10-tetrahydro-6H-azepino[4,5-g]quinoxaline) improved long-term visual episodic/declarative memory as assessed by novel object recognition (NOR) task following oral dosing in normal rats. Memory was assessed by using the three-trial object recognition test. On the first day (exploratory trial), rats were allowed to explore an open arena (44.5×44.5×30.5 cm) for 6 min. On the second day (acquisition trial), rats were allowed to explore the same arena in the presence of two identical objects (both object A) for 3 minutes. On the third day (retention or recall trial), performance was evaluated by allowing the same animal to re-explore the arena for 3 minutes in the presence of two different objects: the familiar object A and a novel object B. An inter-trial interval of 24 hours was imposed between the three NOR trials. Recognition memory was assessed by comparing the time spent exploring a novel (object B) versus a familiar (object A) object during the recall trial. Recognition index was assessed for each animal and expressed as a ratio ((time B/time A+time B)×100).

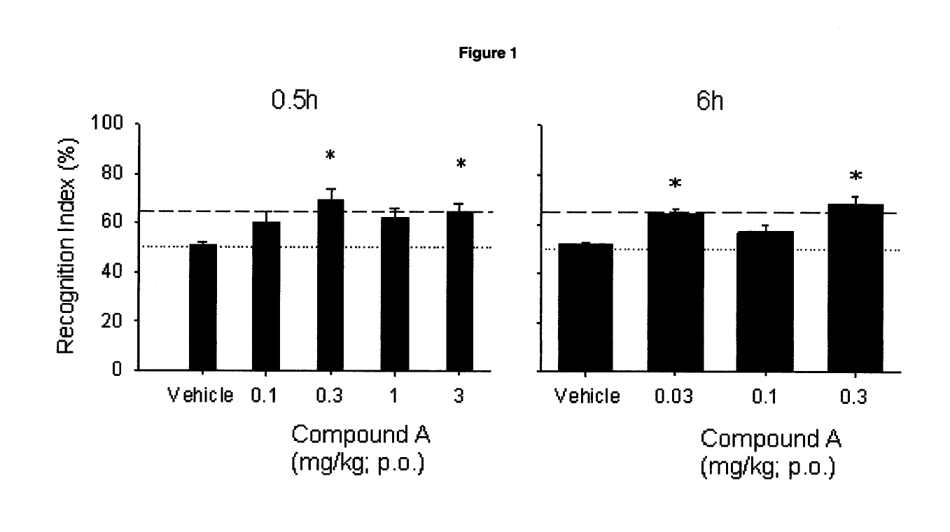

When orally administered 30 minutes before the three trials (i.e., exploratory, acquisition and recall trials), Compound A at 0.3 and 3 mg/kg, (1.5 and 15.1 μmol/kg) p.o., facilitated recognition memory as assessed by enhancement of recognition index when compared to vehicle-treated rats (FIG. 1, left panel). The recognition index of the vehicle-treated group 24 h after the acquisition trial was 50±0.5% demonstrating the inability of this group to recognize the familiar object after this delay (left panel).

Compound A was evaluated for its duration of effect in the NOR task in normal rats. Using similar experimental procedures as described earlier, 0.03, 0.1, and 0.3 mg/kg (0.15, 0.5 and 1.51 μmol/kg) Compound A was orally administered to animals 30 minutes prior to placement in the arena for the exploratory and acquisition trials. For the recall trial (i.e., third day of dosing), animals were placed in the arena at 6 h, post administration. At 0.15 and 1.51 μmol/kg dose levels, Compound A facilitated recognition memory index for up to 6 h after dosing (FIG. 1, right panel). By contrast to the left panel (vehicle-treated), animals treated with Compound A exhibited recognition indexes of 69±5% at the 0.3 mg/kg (1.5 μmol/kg) dose level and 65±3% at the 3 mg/kg (15.1 μmol/kg) dose level.

A follow-up NOR study evaluated the duration of effect of Compound A following a 6 h pre-treatment prior to the recall trial. The recognition index of the vehicle-treated group at 6 h following dosing on the recall trial was 52±0.5% demonstrating the inability of this group to recognize the familiar object after this delay. By contrast, animals treated with Compound A at 0.03 mg/kg (0.15 μmol/kg) and 0.3 mg/kg (1.5 μmol/kg) exhibited recognition indexes of 64±3% and 68±3% respectively; suggesting that the Compound A-treated rats are able to recognize the familiar object for up to 6 h after dosing (right panel).The dashed line at 65% denotes our criteria for biological cognitive enhancing activity. *P<0.05.

### Example 10: In vivo pharmacology: Radial Arm Maze (RAM)

Working memory is a cognitive domain known to be impaired in schizophrenia; the Radial Arm Maze task is commonly used to assess potential cognitive benefit derived from the test compounds. Using this assay, Compound A (7,8,9,10-tetrahydro-6H-azepino[4,5-g]quinoxaline) attenuated cognitive deficits induced by scopolamine in an animal model of working memory. Working memory was assessed in a 3 trial radial arm maze (RAM) task. The RAM task was conducted using an automated eight-arm maze (Med Associates, Inc.) The maze was located on a circular table approximately 88 cm above the floor with overhead lighting in a dedicated testing room and large, high contrast geometric shapes on the wall. Furthermore, additional visual cues were located at the hub entry into each arm, above each the food hopper and on the ceiling. The central platform measured 30.5 cm in diameter with eight arms (9 cm W×45.7 cm L×16.8 cm H) radiating from it. Automatic guillotine doors were located at the entrance to each runway with a pellet receptacle at the distal end of each arm. White noise was audible during all training and testing procedures. Activity on the maze was monitored by tracking quantitative activity (generated by infra-red beam breaks) on the computer interface and monitor screen.

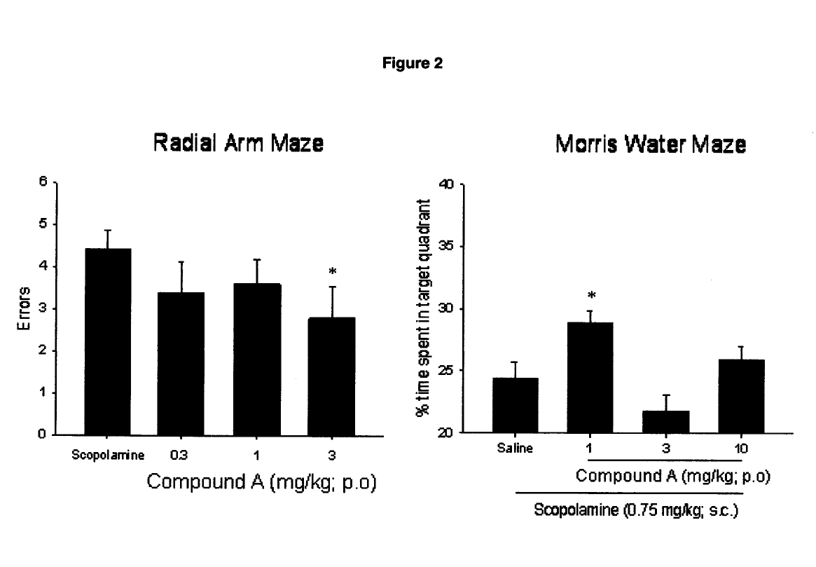

Following the baseline assessment on day 1 and after re-attainment of test session criterion, animals were assessed for their sensitivity to chemically-induced cognitive impairment using the muscarinic antagonist scopolamine (0.2-0.4 mg/kg; s.c.). A dose of scopolamine was determined for each animal based on the minimum dose that produced significant and reliable cognitive impairment. Scopolamine alone or scopolamine plus Compound A (0.3, 1 and 3 mg/kg) (1.5, 5 and 15.1 μmol/kg; p.o.) were administered 0.5 h prior to the acquisition phase trial on day 2 of the protocol. In the acquisition trial, one randomly selected arm was blocked with a Plexiglas barrier situated just inside the arm, behind the hub door. The animal was placed in the central hub of the maze with doors down. After approximately 10 sec, doors to the 7 available arms were raised. The first entry to each open arm was reinforced with a sucrose food pellet. The session ended after all 7 available arms were visited or 5 minutes elapsed. The order of arms visited, reinforcers received, errors (re-entries), time to complete the task, the number of entries and time required to enter 7 available arms and consume food reinforcer were recorded. On day 3 during the recall trial, all 8 arms were available, however, only the first visit to the previously blocked arm (i.e., the arm that was blocked during the acquisition trial) was reinforced. The session ended once the previously blocked arm was visited and the reinforcer was consumed or 5 minutes elapsed. For the recall trial, re-entry errors, the number of (incorrect) arms entered prior to choosing the arm that was blocked during the acquisition trial and the time taken to complete the trial was recorded. The delay between the acquisition and test phase trials was 24 hours. During the acquisition trial in the RAM task, rats were allowed access to 7 of the eight arms whereas, in the test trial, all 8 arms were available, however, only the first visit to the previously blocked arm (i.e., the arm that was blocked during the acquisition trial) was reinforced. Scopolamine (3±0.1 mg/kg; s.c.) was administered 0.5 h prior to the acquisition trial whereas, Compound A (15.1 μmol/kg or 3 mg/kg; p.o.) was administered 0.5 h prior to the test trial. Compound A was able to reverse scopolamine-induced cognitive deficits (left panel). At 3 mg/kg (15.1 μmol/kg dose level, Compound A attenuated scopolamine-induced cognitive deficits (FIG. 2, left panel).

### Example 11: In vivo pharmacology: Morris Water Maze (MWM)

In an animal model of spatial memory, the efficacy of Compound A (7,8,9,10-tetrahydro-6H-azepino[4,5-g]quinoxaline) to attenuate water-maze performances of mice impaired with scopolamine (0.75 mg/kg, s.c.) was evaluated. Following a two-day acclimation period to the maze, mice were trained for 4 days to a platform location in the maze. During the training sessions, each animal was given four trials separated by 5 minutes between each trial. The platform location was constant for each animal. Compound A (1, 3 and 10 mg/kg or 5, 15.1 and 50 μmol/kg, p.o.) was administered 25 minutes prior to each of the four training days. Scopolamine was administered 15 minutes prior to each of the four training days. The probe trial (i.e., no platform present) was conducted on day 5 under drug-free (i.e., water instead of Compound A and saline instead of scopolamine) conditions. In the MWM model, Compound A (1, 3 and 10 mg/kg) was orally administered 25 minutes whereas scopolamine was subcutaneously administered 15 minutes prior to each of the four training days. During training days, each animal was given four trials separated by 5 minutes between trials. The probe trial was conducted on day 5 under drug-free (i.e., water instead of Compound A and saline instead of scopolamine) conditions. Compound A was able to reverse scopolamine-induced cognitive deficits at the 1 mg/kg (5 μmol/kg) dose level (right panel). *P<0.05.Compound A was able to reverse scopolamine-induced cognitive deficits at the 1 mg/kg (5 μmol/kg) dose level (FIG. 2, right panel).

Test compounds for the experiments described herein were employed in free or salt form.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A compound of Formula 1: 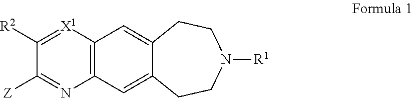
wherein:
X¹ independently is N or CR¹⁰;
independently is H, R¹⁰, OR¹⁰, HNR¹⁰, NR^{10R}¹¹, or halogen;
R¹ independently is H or C₁₋₆ alkyl;
R² independently is H, C₁₋₆ alkyl, aryl, or heteroaryl; which aryl and heteroaryl groups can optionally be substituted with one or more of C₁₋₆ alkyl, halogen, hydroxyl, C₁₋₆ alkoxy, amino, or C₁₋₆ haloalkyl; and
each of R¹⁰ or R¹¹ independently is H, C₁₋₆ alkyl, aryl, or heteroaryl; which aryl and heteroaryl groups can optionally be substituted with one or more of C₁₋₆ alkyl, halogen, hydroxyl, C₁₋₆ alkoxy, amino, or C₁₋₆ haloalkyl;
or a pharmaceutically acceptable salt thereof.

2. (canceled)

3. The compound of claim 1, wherein the compound is a tautomer.

4-5. (canceled)

6. The compound of claim 1, wherein X¹ is N.

7. (canceled)

8. The compound of claim 1, wherein R¹ is H.

9. The compound of claim 1, wherein R¹ is C₁₋₆ alkyl.

10. The compound of claim 1, wherein the compound is selected from:
7,8,9,10-tetrahydro-6H-azepino[4,5-g]quinoxaline; 8-methyl-7,8,9,10-tetrahydro-6H-azepino[4,5-g]quinoxaline; 2-methyl-7,8,9,10-tetrahydro-6H-azepino[4,5-g]quinoxaline; 2,3-dimethyl-7,8,9,10-tetrahydro-6H-azepino[4,5-g]quinoxaline; 2-methyl-3-ethyl-7,8,9,10-tetrahydro-6H-azepino[4,5-g]quinoxaline; 2,3-diethyl-7,8,9,10-tetrahydro-6H-azepino[4,5-g]quinoxaline; 2,8-dimethyl-7,8,9,10-tetrahydro-6H-azepino[4,5-g]quinoxaline; 7,8,9,10-tetrahydro-6H-azepino[4,5-g]quinoxalin-2(1H)-one; 2-chloro-7,8,9,10-tetrahydro-6H-azepino[4,5-g]quinoxaline; 2-chloro-8-methyl-7,8,9,10-tetrahydro-6H-azepino[4,5-g]quinoxaline; 2-methoxy-7,8,9,10-tetrahydro-6H-azepino[4,5-g]quinoxaline; 2-methoxy-8-methyl-7,8,9,10-tetrahydro-6H-azepino[4,5-g]quinoxaline; 2-(N-methylamino)-7,8,9,10-tetrahydro-6H-azepino[4,5-g]quinoxaline; 2-(N,N-dimethylamino)-7,8,9,10-tetrahydro-6H-azepino[4,5-g]quinoxaline; 2-(N-benzylamino)-7,8,9,10-tetrahydro-6H-azepino[4,5-g]quinoxaline; 2-(3-pyridinyl)-7,8,9,10-tetrahydro-6H-azepino[4,5-g]quinoxaline; 7,8,9,10-tetrahydro-6H-azepino[4,5-g]quinoline; 8-methyl-7,8,9,10-tetrahydro-6H-azepino[4,5-g]quinoline; 2-methyl-7,8,9,10-tetrahydro-6H-azepino[4,5-g]quinoline; 2,3-dimethyl-7,8,9,10-tetrahydro-6H-azepino[4,5-g]quinoline; 2,8-dimethyl-7,8,9,10-tetrahydro-6H-azepino[4,5-g]quinoline; 7,8,9,10-tetrahydro-6H-azepino[4,5-g]quinolin-2(1H)-one; 2-chloro-7,8,9,10-tetrahydro-6H-azepino[4,5-g]quinoline; 2-chloro-8-methyl-7,8,9,10-tetrahydro-6H-azepino[4,5-g]quinoline; 2-methoxy-7,8,9,10-tetrahydro-6H-azepino[4,5-g]quinoline; 2-methoxy-8-methyl-7,8,9,10-tetrahydro-6H-azepino[4,5-g]quinoline; 2-(N-methylamino)-7,8,9,10-tetrahydro-6H-azepino[4,5-g]quinoline; 2-(N,N-dimethylamino)-7,8,9,10-tetrahydro-6H-azepino[4,5-g]quinoline; 2-phenyl-7,8,9,10-tetrahydro-6H-azepino[4,5-g]quinoline; 2-(3-pyridinyl)-7,8,9,10-tetrahydro-6H-azepino[4,5-g]quinoline; 1,5,6,7,8,9-hexahydroimidazo[4,5-h][3]benzazepine; 1-methyl-1,5,6,7,8,9-hexahydroimidazo[4,5-h][3]benzazepine; 2-methyl-1,5,6,7,8,9-hexahydroimidazo[4,5-h][3]benzazepine; 1,7-dimethyl-1,5,6,7,8,9-hexahydroimidazo[4,5-h][3]benzazepine; 2,7-dimethyl-1,5,6,7,8,9-hexahydroimidazo[4,5-h][3]benzazepine; 1,2-dimethyl-1,5,6,7,8,9-hexahydroimidazo[4,5-h][3]benzazepine; 2-methyl-1-phenyl-1,5,6,7,8,9-hexahydroimidazo[4,5-h][3]benzazepine; 3,5,6,7,8,9-hexahydroimidazo[4,5-h][3]benzazepin-2(1H)-one; 1-methyl-3,5,6,7,8,9-hexahydroimidazo[4,5-h][3]benzazepin-2(1H)-one; 1-phenyl-3,5,6,7,8,9-hexahydroimidazo[4,5-h][3]benzazepin-2(1H)-one; 1,7-dimethyl-3,5,6,7,8,9-hexahydroimidazo[4,5-h][3]benzazepin-2(1H)-one; and 7-methyl-1-phenyl-3,5,6,7,8,9-hexahydroimidazo[4,5-h][3]benzazepin-2(1H)-one, or a pharmaceutically acceptable salt thereof.

11. The compound of claim 1, wherein the compound is 7,8,9,10-tetrahydro-6H-azepino[4,5-g]quinoxaline or a pharmaceutically acceptable salt thereof.

12. The compound of claim 11, wherein the compound is selected from:
7,8,9,10-tetrahydro-6H-azepino[4,5-g]quinoxaline hydrochloride;
7,8,9,10-tetrahydro-6H-azepino[4,5-g]quinoxaline L-tartrate;
7,8,9,10-tetrahydro-6H-azepino[4,5-g]quinoxaline p-hydroxybenzoate;
and
7,8,9,10-tetrahydro-6H-azepino[4,5-g]quinoxaline succinate, or a solvate thereof.

13. A pharmaceutical composition comprising a compound as claimed in claim 1 and a pharmaceutically acceptable carrier.

14. A method for the treatment or prevention of a disease or condition mediated by a neuronal nicotinic receptor comprising the administration of a compound as claimed in claim 1.

15-23. (canceled)

24. The method of claim 14, wherein the disease or condition is selected from the group of age-associated memory impairment, mild cognitive impairment, age-related cognitive decline, pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, tardive dyskinesia, hyperkinesia, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), and chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, oppositional defiant disorder, Tourette's syndrome, autism, drug and alcohol addiction, tobacco addiction, acute pain, chronic pain, or neuropathies.

25. The method of claim 14, wherein the central nervous system disorder is selected from mild cognitive impairment, age-associated memory impairment, pre-senile dementia, early onset Alzheimer's disease, senile dementia, dementia of the Alzheimer's type, Alzheimer's disease, Lewy Body dementia, micro-infarct dementia, AIDS-related dementia, HIV-dementia, multiple cerebral infarcts, Parkinsonism, Parkinson's disease, Pick's disease, progressive supranuclear palsy, Huntington's chorea, tardive dyskinesia, hyperkinesia, mania, attention deficit disorder, attention deficit hyperactivity disorder, anxiety, depression, dyslexia, schizophrenia, cognitive dysfunction in schizophrenia, depression, obsessive-compulsive disorders, or Tourette's syndrome.

26. The method of claim 14, wherein the central nervous system disorder is selected from Alzheimer's disease, mania, attention deficit disorder, attention deficit hyperactivity disorder, anxiety, dyslexia, schizophrenia, cognitive dysfunction in schizophrenia, depression, obsessive-compulsive disorders, or Tourette's syndrome.

27. A compound 7,8-diamino-2,3,4,5-tetrahydro-1H-benzo[d]azepine.

28. A compound 7,8-diamino-3-trifluoroacetyl-2,3,4,5-tetrahydro-1H-benzo[d]azepine or tert-butyl 7,8-diamino-1,2,4,5-tetrahydro-3H-3-benzazepine-3-carboxylate.

29. A method of making a 3-N-protected-7,8-diamino-2,3,4,5-tetrahydro-1H-benzo[d]azepine, comprising the steps of:
i) nitrating 2,3,4,5-tetrahydro-1H-3-benzazepine to form 7-nitro-2,3,4,5-tetrahydro-1H-3-benzazepine;
ii) converting the 7-nitro-2,3,4,5-tetrahydro-1H-3-benzazepine into a suitable 3-N-protected derivative;
iii) reducing the 7-nitro group to form a 7-amino group;
iv) converting the 7-amino group to an amide derivative;
v) nitrating the 7-acylamino compound to form the 7-acylamino-8-nitro compound;
vi) reducing the 8-nitro group to form an 8-amino group; and
vii) hydrolyzing the acyl group from the 7-position amine.

30. A method of making a compound of claim 1, comprising condensation of a 3-N-protected-7,8-diamino-2,3,4,5-tetrahydro-1H-benzo[d]azepine with one or more reagents.

31. The method of claim 30, further comprising the step of removing the protecting group from the 3-position amino group.
